(19) United States
(12) Patent Application Publication
(10) Pub. No.: US 2023/0103883 A1
(43) Pub. Date: Apr. 6, 2023

(54) METHODS AND APPARATUSES FOR PERITONEAL CATHETER PLACEMENT

(71) Applicants: Baxter International Inc., Deerfield, IL (US); Baxter Healthcare SA, Glattpark (Opfikon) (CH)

(72) Inventors: Shawn Collin Oppegard, Fox River Grove, IL (US); Unberto Garcia, Bellwood, IL (US); Jorge Augusto Del Castillo, Des Plaines, IL (US); Kent Alan Williams, Chicago, IL (US); Carlos Alberto Corrales, Vernon Hills, NY (US); Mary Kate Nowak, Burr Ridge, IL (US); Shashanka Belur Shanthakumar Jain, Karnataka State (IN); Kedar Kulkarni, Karnatake State (IN); James Mitchell White, Grayslake, IL (US); Brian James Connell, Evanston, IL (US); Leah Ann Swanson, McHenry, IL (US); Nicole Alexandra Chen, Long Grove, IL (US); Eajer Toh, Sugar Land, TX (US); Joanna Rita Yanaga, Arlington Heights, IL (US)

(21) Appl. No.: 17/961,249

(22) Filed: Oct. 6, 2022

Related U.S. Application Data
(60) Provisional application No. 63/252,924, filed on Oct. 6, 2021.

(51) Int. Cl. A61M 25/06; A61M 25/09; A61M 1/28

(52) U.S. Cl. CPC A61M 25/065; A61M 25/09041; A61M 1/285; A61M 2205/273; A61B 2017/3413

(57) ABSTRACT

Methods and apparatuses for peritoneal catheter placement are disclosed herein. An example method includes using a puncture device to create an access hole to a patient's peritoneal cavity. The puncture device includes a lumen that enables a guidewire to be inserted into the peritoneal cavity. The puncture device is then replaced with a catheter, which is inserted along the guidewire. A surgical seal is next applied to connect the catheter to adjacent patient muscle tissue. After the guidewire is removed, a tunneling device creates a pathway through fat tissue to anchor the catheter. An end of the tunneling device is aligned with an external piercing device to pierce the patient's skin from the outside, creating a catheter exit hole. An end of the catheter is connected to the tunneling device and pulled through the exit hole, thereby enabling the catheter to be connected to peritoneal dialysis tubing for a treatment.

### PRIORITY CLAIM

This application claims priority to and the benefit as a non-provisional application of U.S. Patent Application Ser. No. 63/252,924, filed Oct. 6, 2021, the entire disclosure of which is hereby incorporated by reference.

### BACKGROUND

Due to various causes, a person's renal system can fail. Renal failure produces several physiological derangements. For instance, it is no longer possible for a person with renal failure to balance water and minerals or to excrete daily metabolic load. Additionally, toxic end products of metabolism, such as, urea, creatinine, uric acid, and others, may accumulate in a patient's blood and tissue.

Reduced kidney function and, above all, kidney failure is treated with dialysis. Dialysis removes waste, toxins, and excess water from the body that normal functioning kidneys would otherwise remove. Dialysis treatment for replacement of kidney functions is critical to many people because the treatment is lifesaving.

One type of kidney failure therapy is peritoneal dialysis (“PD”), which infuses a dialysis solution, also called dialysis fluid or PD fluid, into a patient's peritoneal cavity via a catheter. The dialysis fluid contacts a peritoneal membrane in a patient's peritoneal cavity. Waste, toxins, and excess water pass from the patient's bloodstream, through the capillaries in the peritoneal membrane, and into the dialysis fluid due to diffusion and osmosis (i.e., an osmotic gradient occurs across the membrane). An osmotic agent in the dialysis fluid provides the osmotic gradient. Used or spent dialysis fluid is drained, removing waste, toxins, and excess water from the patient. This cycle is repeated multiple times.

There are various types of peritoneal dialysis therapies, including continuous ambulatory peritoneal dialysis (“CAPD”), automated peritoneal dialysis (“APD”), tidal flow dialysis, and continuous flow peritoneal dialysis (“CFPD”). CAPD is a manual dialysis treatment. Here, the patient manually connects an implanted catheter to a drain to allow used or spent dialysis fluid to drain from the peritoneal cavity. The patient then switches fluid communication so that the patient catheter communicates with a bag of fresh dialysis fluid to infuse the fresh dialysis fluid through the catheter and into the patient. The patient disconnects the catheter from the fresh dialysis fluid bag and allows the dialysis fluid to dwell within the peritoneal cavity, where the transfer of waste, toxins, and excess water takes place. After a dwell period, the patient repeats the manual dialysis procedure, for example, four times per day. Manual peritoneal dialysis requires a significant amount of time and effort from the patient, leaving ample room for improvement.

Automated peritoneal dialysis (“APD”) is similar to CAPD in that the dialysis treatment includes drain, fill, and dwell cycles. APD machines, however, perform the cycles automatically, typically while the patient sleeps. APD machines free patients from having to manually perform the treatment cycles and from having to transport supplies during the day. APD machines connect fluidly to an implanted catheter, to a source or bag of fresh dialysis fluid, and to a fluid drain. APD machines pump fresh dialysis fluid from a dialysis fluid source, through the catheter, and into the patient's peritoneal cavity. APD machines also allow for the dialysis fluid to dwell within the peritoneal cavity and for the transfer of waste, toxins, and excess water to take place. The source may include multiple liters of dialysis fluid including several solution bags.

APD machines pump used or spent dialysate from the patient's peritoneal cavity, though the catheter, and to the drain. As with the manual process, several drain, fill, and dwell cycles occur during dialysis. A “last fill” may occur at the end of the APD treatment. The last fill fluid may remain in the peritoneal cavity of the patient until the start of the next treatment, or may be manually emptied at some point during the day.

For PD, a catheter may be inserted into a patient's peritoneal cavity using a trocar, guidewire, dilators, sheath, and/or a needle. The trocar includes an obturator and a lumen. When used, the trocar and obturator pierce through the patient's skin, fat tissue, and muscle through an access hole into the peritoneal cavity. After piercing, a guidewire is inserted through the trocar. When the guidewire is placed into a desired position, the trocar is removed and replaced with a catheter. Oftentimes, the access hole has to be enlarged to accommodate the catheter. After initial catheter insertion, the catheter is sometimes tunneled through the patient's fat tissue to provide connection stability and reduce post-operative leaks.

The known PD catheter insertion steps can take a number of minutes for a clinician to complete and often result in a relatively large access hole. Additionally, these known catheter placement steps risk puncturing a patient's peritoneal wall with a surgical tool used to pierce into the peritoneal cavity. Further, the surgical tools used for placing the catheter can be difficult for relatively new operators.

A need accordingly exists for an improved catheter placement method.

### SUMMARY

An improved method and apparatuses are disclosed for catheter placement for peritoneal dialysis (“PD”). The example method disclosed herein includes relatively few steps compared to known catheter placement methods that can include up to seven or eight separate steps. As discussed herein, during a first step, a patient's skin, fat tissue, and muscle are dissected or otherwise punctured to create an access hole into a peritoneal cavity. The puncture may be made with a puncture device having a retractable blunt tip. The puncture device includes, for example, a single or dual lumen safety needle and/or a single or dual lumen trocar. The retractable blunt tip retracts to expose a sharp tip for piercing through skin, fat tissue, and/or muscle to reach the peritoneal cavity. However, the blunt tip actuates to cover the sharp tip upon entry into the peritoneal cavity to prevent the sharp tip from inadvertently piercing the peritoneal wall.

During a second step, a guidewire is routed through the puncture device and into the patient's peritoneal cavity. In some instances, an endoscope is also routed though the puncture device (or through another access hole) to provide internal visualization for a clinician to set the guidewire. After the guidewire is set, the puncture device is removed leaving the guidewire in place. As disclosed herein, a diameter of the puncture device may approximate a diameter of a catheter. Using a similar diameter enables the catheter to be inserted into the patient without having to expand the access hole. A clinician is accordingly able to easily insert the catheter (using a sheath) along the guidewire to the set location during this second step without having to further cut or slice patient tissue. If a diameter of the puncture device is significantly smaller, a dilator and/or sheath may be needed for catheter insertion.

During a third step of the example method, a leak mitigation seal is placed between the catheter and the patient's muscle tissue at the access hole. During this step, a surgical sealant is used to reinforce closure around the catheter to prevent fluid leakage from the peritoneal cavity. The sealants may include fibrin, albumin, synthetic sealants, or combinations thereof. In some instances, the leak mitigation may occur after tunneling.

A fourth step of the example method includes using a tunneling device to route a portion of the catheter that is outside of the peritoneal cavity through fat tissue of the patient (under the skin). The tunneling of the catheter helps anchor or otherwise secure the catheter to the patient while preventing post-procedure leaks. The tunneling device includes a retractable sharp tip for puncturing through skin. The tunneling device also includes a removable handle that enables the catheter to be connected. This connection enables the catheter to be pulled through the created fat tunnel and out through the patient's skin via the tunneling device. In some embodiments, a piercing tool may be used in conjunction with a tunneling device (having a blunt tip) to aid in alignment and piercing of the patient's skin from the outside while the blunt tip of the tunneling device provides pressure from the inside.

In light of the disclosure set forth herein, and without limiting the disclosure in any way, in a first aspect of the present disclosure, which may be combined with any other aspect, or portion thereof, described herein a catheter insertion method includes causing an access hole to be created through skin, fat tissue and muscle to a peritoneal cavity of a patient using a puncture device having a first diameter, the puncture device including a retractable blunt tip that exposes a sharp tip when retracted, causing a guidewire to be routed into the peritoneal cavity through the puncture device after the blunt tip is extended past the sharp tip, causing the puncture device to be removed after the guidewire is routed into the peritoneal cavity, causing a first end of a catheter to be inserted through the access hole into the peritoneal cavity along the guidewire, the catheter having a second diameter, causing the guidewire to be removed after insertion of the first end of the catheter, causing a surgical sealant to create a seal between a portion of the catheter and adjacent muscle at the access hole, causing a tunneling device to create a pathway through the fat tissue under the skin of the patient, the tunneling device including a first end with a retractable sharp tip or a blunt tip and a second end connected to a removable handle, the second end connectable to a second end of the catheter to pull the catheter through the pathway when the handle is removed, causing a piercing tool having a needle in a middle of an alignment cup to align with the first end of the tunneling device to sandwich a portion of the skin therebetween, causing the needle of the piercing tool to pierce the portion of the skin externally to create a catheter exit hole, causing the tunneling device and the second end of the catheter to exit the patient at the catheter exit hole, and causing the second end of the catheter to be connected to peritoneal dialysis tubing for a peritoneal dialysis treatment.

In a second aspect of the present disclosure, which may be combined with any other aspect listed herein, the puncture device includes at least one of a mosquito hemostat, a single safety needle, a dual lumen safety needle, single lumen trocar, or a duel lumen trocar.

In a third aspect of the present disclosure, which may be combined with any other aspect listed herein, the blunt tip of the puncture device is configured to cover the sharp tip after the puncture device creates the access hole to the peritoneal cavity.

In a fourth aspect of the present disclosure, which may be combined with any other aspect listed herein, the first diameter of the puncture device and the second diameter of the catheter is between 3 millimeters (“mm”) and 15 mm.

In a fifth aspect of the present disclosure, which may be combined with any other aspect listed herein, the first diameter of the puncture device is equal to the second diameter of the catheter.

In a sixth aspect of the present disclosure, which may be combined with any other aspect listed herein, the first diameter of the puncture device is between 0.5 mm and 3 mm shorter than the second diameter of the catheter.

In a seventh aspect of the present disclosure, which may be combined with any other aspect listed herein, the surgical sealant includes at least one of fibrin, albumin, a synthetic sealant, or combinations thereof.

In an eighth aspect of the present disclosure, which may be combined with any other aspect listed herein, the puncture device includes a first lumen to enable the guidewire the pass through and a second lumen to enable an endoscope to pass through to the peritoneal cavity to provide visualization for placement of the guidewire.

In a ninth aspect of the present disclosure, which may be combined with any other aspect listed herein, the catheter insertion method further includes causing a sheath or a dilator to be inserted into the access hole after the puncture device is removed for catheter insertion.

In a tenth aspect of the present disclosure, which may be combined with any other aspect listed herein, a safety needle apparatus for insertion of a peritoneal dialysis catheter includes a shaft including a first end and a second end, the first end including a sharp bevel, a handle connected to the second end of the shaft, the handle including an engagement lever moveable between a lock position and an unlock position, a guidewire lumen including a first end and a second end, the first end being aligned with the first end of the shaft, the guidewire lumen configured to receive a guidewire for placement of the peritoneal dialysis catheter, a blunt tip located within the shaft at the first end, the blunt tip connected to the engagement lever of the handle by a retraction mechanism, wherein the engagement lever of the handle is configured to prevent retraction of the blunt tip into the shaft when the engagement lever is moved to the lock position.

In an eleventh aspect of the present disclosure, which may be combined with any other aspect listed herein, the retraction mechanism includes a spring that causes the blunt tip to extend past the sharp bevel when the engagement lever is moved from a sharp bevel exposure position to a blunt tip exposure position.

In a twelfth aspect of the present disclosure, which may be combined with any other aspect listed herein, moving the engagement lever from the blunt tip exposure position to the sharp bevel exposure position enables the spring to be pushed into a compressed state from resistance of the blunt tip contacting tissue.

In a thirteenth aspect of the present disclosure, which may be combined with any other aspect listed herein, the shaft has a diameter that is equal to or within 3 millimeters of a diameter of the peritoneal dialysis catheter.

In a fourteenth aspect of the present disclosure, which may be combined with any other aspect listed herein, the first end of the shaft includes an echogenic texture for ultrasound visualization.

In a fifteenth aspect of the present disclosure, which may be combined with any other aspect listed herein, the handle includes a sharp bevel indicator that is configured to provide a visual indication when the engagement lever is in a sharp bevel exposure position.

In a sixteenth aspect of the present disclosure, which may be combined with any other aspect listed herein, the guidewire lumen is connected to an exterior of the shaft and an exterior of the handle.

In a seventeenth aspect of the present disclosure, which may be combined with any other aspect listed herein, the guidewire lumen is positioned inside the shaft and the handle.

In an eighteenth aspect of the present disclosure, which may be combined with any other aspect listed herein, at least a portion of the blunt tip includes a transparent material, and the blunt tip includes an endoscope camera.

In a nineteenth aspect of the present disclosure, which may be combined with any other aspect listed herein, the endoscope camera is communicatively coupled to at least one of a tablet computer, a smartphone, or workstation, or a display monitor.

In a twentieth aspect of the present disclosure, which may be combined with any other aspect listed herein, the blunt tip includes a blunt obturator or an inner shaft and the sharp bevel includes a sharp cannula or an outer shaft.

In a twenty-first aspect, any of the features, functionality and alternatives described in connection with any one or more of FIGS. 2 to 28 may be combined with any of the features, functionality and alternatives described in connection with any other of FIGS. 2 to 28.

In light of the present disclosure and the above aspects, it is therefore an advantage of the present disclosure to provide a catheter insertion method that includes less steps and increased safety and ease of use compared to known catheter insertion procedures.

It is another advantage of the present disclosure to provide a piercing device, a tunneling device, and/or a piercing tool to perform the example catheter insertion method in less steps than known catheter insertion procedures.

It is yet another advantage of the present disclosure to provide a piercing device that enables an endoscope to be inserted through the same patient access hole as a guidewire to reduce a number of puncture locations on a patient.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

FIG. 1 shows a known catheter insertion procedure.

FIG. 2 shows an example procedure for a catheter insertion method disclosed herein, according to an example embodiment of the present disclosure.

FIG. 3 is a diagram illustrative of how an access hole is created using a puncture device, according to an example embodiment of the present disclosure.

FIG. 4 is a diagram of a catheter inserted into the access hole of FIG. 3, according to an example embodiment of the present disclosure.

FIGS. 5 to 9 are diagrams of example puncture devices, according to example embodiments of the present disclosure.

FIGS. 10 to 13 are diagrams of a puncture device of FIGS. 5 to 9 operating in conjunction with an endoscope or other visualization instrument, according to example embodiments of the present disclosure.

FIGS. 14 to 17B are diagrams of a tunneling device, according to an example embodiment of the present disclosure.

FIG. 18 is a diagram of a piercing tool, according to an example embodiment of the present disclosure.

FIG. 19 is a diagram that shows how the piercing tool of FIG. 18 is used with a tunneling device to create a catheter exit hole, according to an example embodiment of the present disclosure.

FIG. 20 is a diagram showing a blunt tunneling device and the piercing tool of FIG. 10, according to an example embodiment of the present disclosure.

FIGS. 21 to 27 are diagrams showing alternative embodiments of a tunneling device, according to example embodiments of the present disclosure.

FIG. 28 is a diagram of a guiding stylet, according to an example embodiment of the present disclosure.

### DETAILED DESCRIPTION

An example catheter insertion method for peritoneal dialysis (“PD”) is disclosed herein. Additionally, surgical tools for performing the example PD catheter insertion method are also disclosed herein. The surgical tools may include a puncture device for creating an initial access hole through a patient's skin, fat tissue, and muscle to a peritoneal cavity. The puncture device enables a guidewire to pass through and includes a retractable blunt tip to prevent inadvertently piercing or tearing the patient's peritoneal wall. The puncture device has a diameter that is approximately equal to or slightly shorter than (e.g., 0 to 3 millimeters (“mm”)) than a diameter of a catheter. In some instances, the puncture device may have a diameter that is up to 6 mm longer than a diameter of the catheter. This enables a catheter to be inserted into the patient (along the guidewire) after the puncture device is removed without having to spend additional time expanding the patient's access hole.

In some embodiments, a dilator and/or sheath may be used for catheter insertion along a guidewire. In these embodiments, the dilator and/or sheath is inserted along the guidewire after the puncture device is removed. The catheter may then be inserted through the sheath along the guidewire to a desired placement.

In addition to a puncture device, the example method disclosed herein may be performed with a tunneling device. As disclosed herein, the tunneling device is configured to create a pathway under a patient's skin through the fat tissue to anchor or otherwise secure the catheter to the patient. An end of the catheter (opposite of the catheter end that is located in the peritoneal cavity) is connected to an end of the tunneling device to be pulled through the fat tissue. The tunneling device may include a retractable sharp tip to help create a catheter exit hole. In some embodiments, the method includes using a piercing tool with a needle placed in a middle of an alignment cup. The end of the tunneling device aligns with the alignment cup, sandwiching a portion of a patient's skin therebetween. The opposing blunt tip of the tunneling device provides pressure and support to enable the needle of the piercing tool to pierce through a patient's skin to create the catheter exit hole. After the exit hole is created, the tunneling device and the end of the catheter are pulled through. The tunneling device is disconnected from the catheter, enabling the catheter to be connected to peritoneal dialysis (“PD”) tubing to perform a PD treatment.

The example method disclosed herein is more efficient than known catheter insertion methods. The surgical tools disclosed herein, including the puncture device, the tunneling device, and the piercing tool help reduce the number of procedural steps compared to the known catheter insertion methods. The example tools disclosed herein also include retractable tips that prevent inadvertent piercing of patient tissue, such as the peritoneal wall.

The example method is discussed herein with reference to peritoneal dialysis. It should be appreciated that the example method and related surgical tools may be used for other medical applications for inserting a catheter or similar tube. For example, the method may be used for gastric procedures, bladder procedures, lung procedures, etc.

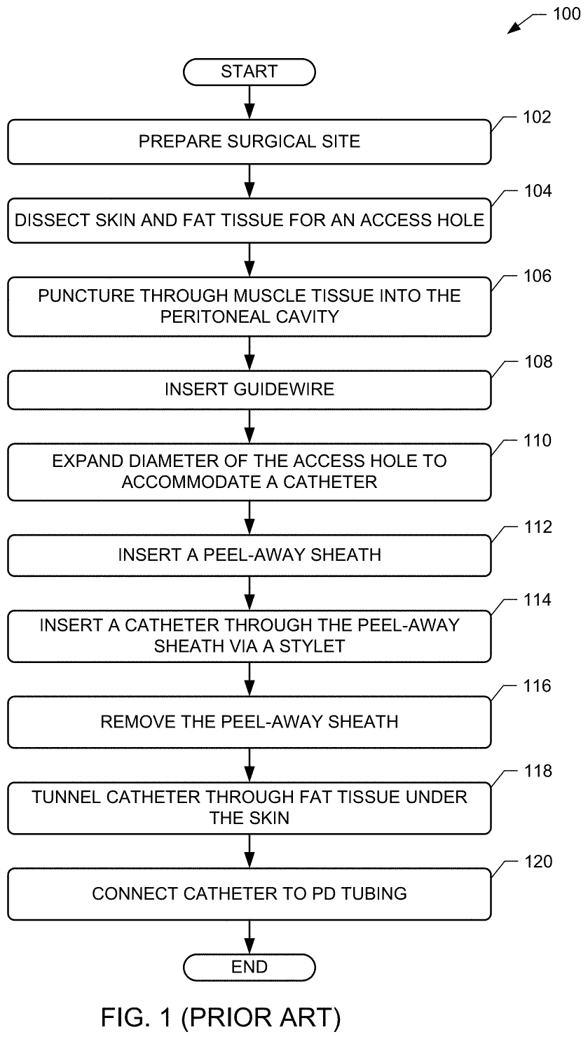

FIG. 1 shows a known catheter insertion procedure 100. The procedure 100 may be carried out by a radiologist, a nephrologist, or other clinician using a percutaneous Seldinger technique. The procedure 100 is performed to insert a catheter for PD including CAPD, APD, and/or end-stage renal disease treatments. The procedure 100 begins when a surgical site is located on a patient's abdomen (block 102). The surgical site is cleaned, disinfected, and applied a local anesthetic.

After the surgical site is prepared, the patient's skin and fat tissue are dissected using a scalpel and/or hemostat or punctured using a basic sharp needle or a blunt needle (e.g., a Hawkins blunt needle) (block 104). The same instrument or a safety needle is then used to puncture through muscle tissue into the peritoneal cavity (block 106). In some instances, the larger force needed to pierce through the muscle tissue can cause the sharp needle to pierce or perforate internal organs including the peritoneal wall. To prevent these undesired piercings, a clinician may instead use a veress safety needle, which has a retractable blunt tip. The veress safety needle deploys the retractable tip after the needle reaches the peritoneal cavity to cover a sharp tip used for muscle piercing. In some instances, a clinician may use an ultrasound device to confirm the tip of the veress safety needle is fully inside the peritoneal cavity or located in pre-peritoneal space.

After the access hole is formed, a guidewire is inserted (block 108). In the known procedure, 100, the safety needle or other puncture device is removed and replaced with a trocar or a dilator having a single lumen. The guidewire is routed through the trocar and to a desired location. In some instances, a clinician may create a second access hole for an endoscope to provide visualization for placement of the guidewire. Once the guidewire is in position, the trocar is removed.

Oftentimes, the trocar has a relatively small diameter, such as 3 to 5 mm. However, a catheter usually has a larger diameter to enable larger volumes of fluid to be added to and removed from the peritoneal cavity for PD treatments. The use of larger diameter catheters prevents waste materials in used dialysis fluid from clogging or partially blocking fluid flow. To accommodate insertion of a catheter, a diameter of the access hole is increased (block 110). The access hole is dilated or expanded using an incision tool such as a sharp scalpel or two or more dilators of increasing size.

After the diameter of the access hole is increased, the example procedure 100 continues when a peel-away sheath is inserted (block 112). The sheath serves as a conduit for the catheter. The catheter is then inserted along the guidewire and through the sheath (block 114). Specifically, the guidewire is placed through the catheter as the catheter is inserted into the access hole and into the sheath. In some instances, a rigid stylet is used for catheter insertion. The stylet provides rigidity for the catheter as it is inserted into the sheath through the access hole. The sheath is then peeled away and removed from the patient, thereby leaving the catheter in the patient (block 116). At this point, a first end of the catheter is positioned inside the peritoneal cavity as directed by the guidewire and a second, opposing end of the catheter is protruding from the patient.

To help anchor or connect the catheter to the patient, the second end is tunneled by a clinician through a layer of fat underneath the skin (block 118). This tunneling also helps reduce fluid leaks once the access hole is closed. In some instances, a tunneling device is used to form a pathway through the fat tissue. To provide a catheter exit, the clinician often uses a puncture needle or sharp scalpel to pierce the patient's skin at an exit location, which is located away from the access hole. Once the catheter exit hole is formed, the second end of the catheter is pushed out of the skin to protrude from the patient. After cleaning, the second end of the catheter may be connected to tubing for a PD treatment (block 120). The procedure 100 then ends.

A known issue with the above procedure 100 is the number of steps. The relatively large number of steps can be time consuming to perform. Additionally, the large number of steps can be troublesome for newer clinicians, where one miss-step can lead to an unintentional patient puncture, fluid leakage, infection, and/or patient discomfort. Moreover, these steps are either performed blindly inside the patient or using an endoscope that has to be inserted through another access hole, which further increases procedure time and has its own recovery complications.

The example catheter insertion method disclosed herein eliminates some of the steps described above in connection with the procedure 100. For instance, the disclosed catheter insertion method uses a single puncture device that includes at least one lumen. The puncture device also has a retractable blunt tip that covers a sharp tip. These features enable a single puncture device to pierce skin, fat tissue, and muscle for creating an access hole to a patient's peritoneal cavity. Further, instead of using a separate device with a lumen, the puncture device includes a lumen to enable a guidewire to be quickly inserted. In some instances, the example puncture device disclosed herein may also include a second lumen to enable an endoscope to pass through, thereby eliminating the need for a second access hole into the peritoneal cavity. In addition to above, the puncture device has a diameter that is equal to or approximately equal to a diameter of a catheter (e.g., within 2-6 mm of the catheter diameter). This means a catheter may be inserted after the puncture device is removed without having to first increase the sides of the access hole.

The example catheter insertion method also uses a tunneling device with a retractable sharp tip. This enables the tunneling device to be used to safely tunnel through a fat layer under the skin. A sharp tip of the tunneling device may then be extended to pierce through skin to create the catheter exit port. The tunneling device disclosed herein includes a removable handle to enable the second end of the catheter to be connected. A clinician may tunnel the catheter by pulling the tunneling device out of the catheter exit hole, causing the second end of the catheter to follow.

In some instances, to form the exit hole, a piercing tool (located on an outside of the patient) aligns with a blunt tip of the tunneling device (located underneath the patient's skin) to pinch or sandwich the patient's skin therebetween. The use of the piercing tool prevents a clinician from accidently piercing their hand within the tunneling when exiting the skin. The piercing tool also creates a smaller catheter exit hole for a tighter seal around the catheter.

### Example Catheter Insertion Method

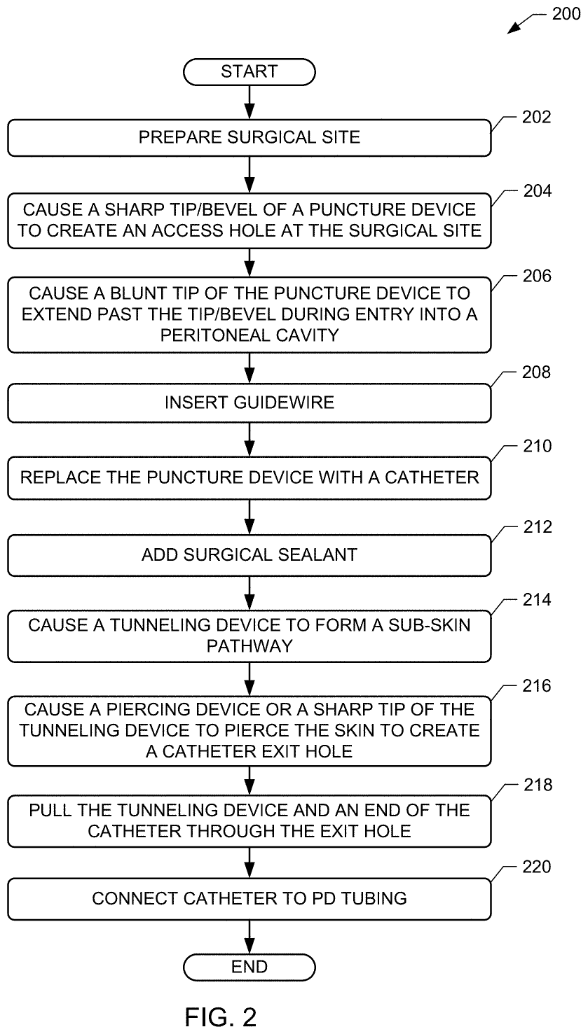

FIG. 2 shows an example procedure 200 for the catheter insertion method disclosed herein, according to an example embodiment of the present disclosure. Although the procedure 200 is described with reference to the flow diagram illustrated in FIG. 2, it should be appreciated that many other methods of performing the steps associated with the procedure 200 may be used. For example, the order of many of the blocks may be changed, certain blocks may be combined with other blocks, and many of the blocks described may be optional. In an embodiment, the number of blocks may be changed. For example, different steps may be performed based on which type of puncture device, piercing tool, and/or tunneling device are used. The example procedure 200 has fewer steps compared to traditional catheter placement procedures, which were discussed above in connection with FIG. 1.

The example procedure 200 begins when a surgical site is prepared (block 202). The surgical site may be located in proximity to a patient's peritoneal cavity and be prepared through sterilization and application of a local anesthesia. Next, an access hole is created at the surgical site through skin, fat tissue, and muscle to a peritoneal cavity (block 204). The access hole is created via a puncture device. In some instances, a dissection tool may be used first to dissect skin tissue.

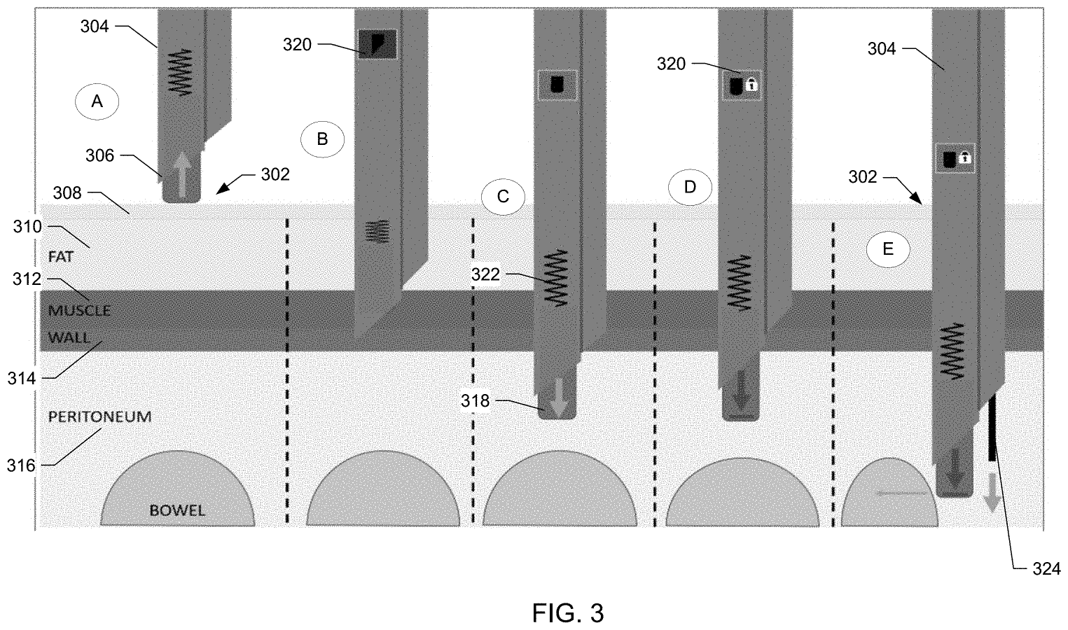

FIG. 3 is a diagram illustrative of how an access hole 302 is created using a puncture device 304, according to an example embodiment of the present disclosure. The puncture device 304 includes a sharp tip/bevel 306 that is configured to incise or otherwise pierce through skin tissue 308, fat tissue 310, muscle tissue 312, and/or a peritoneal wall 314 to form the access hole 302 (shown in Events A and B). In some instances, the puncture device 304 may be used after the skin tissue is dissected. At Event C, the puncture device 304 reaches the peritoneal cavity 316 (e.g., the peritoneum). At this point, a blunt tip 318 of the puncture device 304 is extended or deployed past the sharp/bevel tip 306 (block 206 of FIG. 2). In the embodiment shown in FIG. 3, the blunt tip 318 is in a retracted position for Events A and B, which enables the sharp tip/bevel 306 to be a leading edge for the puncture device 304. However, at Event C, the blunt tip 318 becomes the leading edge, which prevents the sharp tip 306 from accidently puncturing the peritoneal wall 314 and/or other tissue organs including a patient's bowel.

FIG. 3 also shows that the puncture device 304 includes a sharp tip indicator 320. When the blunt tip 318 is in a retracted position, the sharp tip indicator 320 at Event B shows the sharp tip/bevel 306 is exposed. The indicator 320 provides a reminder to a clinician as to whether the sharp tip/bevel 306 is exposed when the clinician may not be able to view the end of the puncture device 304. At Event C when the blunt tip 318 is extended, the indicator 320 changes to specify that the sharp tip/bevel is no longer exposed.

As shown in FIG. 3, the blunt tip 318 is connected to a retraction mechanism 322, which may be connected to an engagement lever (e.g., a switch) in a handle (not shown). Actuation of the engagement lever (e.g., releasing the switch or moving from a sharp tip/bevel exposure position to a blunt tip exposure position) enables a spring of the retraction mechanism 322 to compress and pull the blunt tip 318 upwards into the puncture device 304, thereby retracting the blunt tip 318 from resistance applied by the tissue 308 to the peritoneal wall 314, as shown in Events A and B. The unlocking of the engagement lever enables the automatic retraction to occur. In Event C, after the exposed sharp tip/bevel 306 has pierced through the tissue 308 to 314, the lack of resistance causes the retraction mechanism 322 to expand, thereby pushing the blunt tip 318 outward to prevent the sharp tip/bevel 306 from being accidently exposed during subsequent use of the puncture device 304.

As illustrated at Event D of FIG. 3, the indicator 320 shows that the engagement lever is engaged. The engagement lever may be located in a handle of the puncture device 304 and is configured to move between a lock position and an unlock position. When in the lock position, the engagement lever prevents the retraction mechanism 322 from being moved, thereby preventing the blunt tip 318 from being retracted when force is applied from the tissue. The engagement lever accordingly prevents the sharp tip/bevel 306 from becoming a leading edge when a significant amount of force is applied on the blunt tip 318, thereby preventing accidental puncturing of a patient's peritoneal membrane wall or bowel. In contrast, in Event A when the engagement lever is engaged, force from contacting the skin 308 causes the blunt tip 318 to move to the retract position, exposing the sharp tip/bevel 306.

As shown in FIG. 3 at Event E, after the puncture device 304 is positioned in a desired location/depth in the patient's peritoneal cavity, a guidewire 324 is inserted (block 208 of FIG. 2). The puncture device 304 may include a separate lumen for receiving the guidewire 324. This configuration enables the same device that creates the access hole 302 to also be used for inserting the guidewire 324 instead of using another device. The extension of the blunt tip 318 prevents patient tissue from being cut or pierced even when the guidewire 324 is being positioned. In other embodiments, a dilator and/or sheath may be inserted after the puncture device 304 is removed to enable catheter insertion.

Returning to FIG. 2, after the guidewire 324 is positioned, the puncture device 304 is removed from the access hole 302 and replaced with a catheter using a sheath (block 210). In some embodiments, the puncture device 304 has a first diameter and the catheter has a second diameter, each diameter being between 3 millimeters (“mm”) and 15 mm, for example. In some instances, the first diameter of the puncture device 304 is equal to the second diameter of the catheter. Alternatively, the first diameter of the puncture device 304 is between 0.5 mm and 6 mm shorter (or longer) than the second diameter of the catheter. A dilator and/or sheath may be used for catheter insertion.

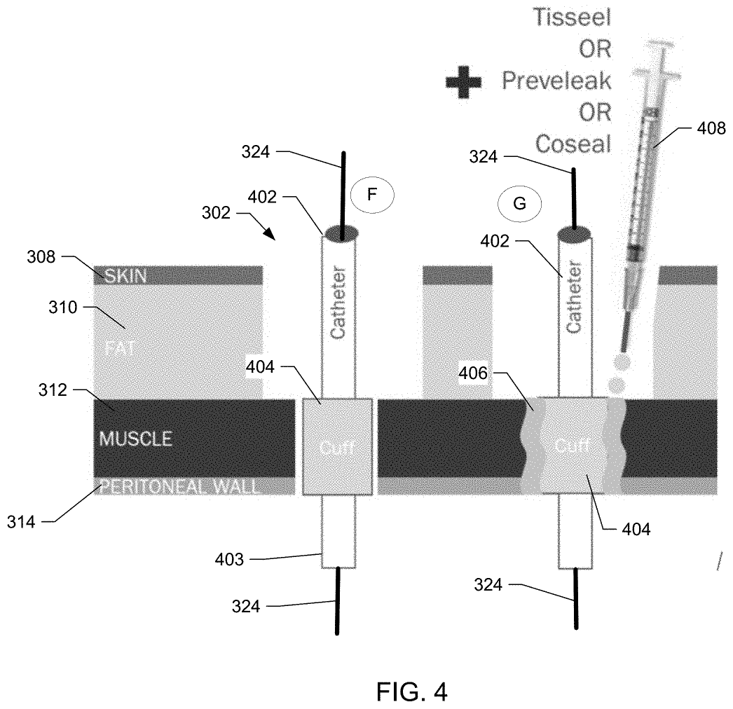

FIG. 4 is a diagram of a catheter 402 inserted into the access hole 302, according to an example embodiment of the present disclosure. A first end 403 of the catheter 402 is placed into the access hole 302 along the guidewire 324. The catheter 402 is positioned such that a cuff 404 is aligned with adjacent muscle tissue 312, as shown at Event F. Next at Event G, a surgical sealant 406 is applied (block 212 of FIG. 2). The surgical sealant 406 may include fibrin (e.g., Tisseel™), albumin (e.g., Preveleak™), a synthetic sealant (e.g., Coseal™), or combinations thereof. The surgical sealant 406 may also include mucoadhesives, cyanoacrylates, chitosan-based, expanding foams/sponges, algae-mimetic adhesives, extracellular matrix hydrogels, etc. The surgical sealant 406 is dispensed from a dispensing device 408, such as a syringe. The surgical sealant 406 creates a fluid-tight seal between the cuff 404 and the adjacent muscle tissue 312 to prevent post-operative dialysate leaks during peritoneal dialysis. After the surgical sealant 406 is applied, the guidewire 324 may be removed through the catheter 402.

Returning to FIG. 2, the example procedure 200 continues by causing a tunneling device to create a pathway under the skin tissue 308 through the fat tissue 310 (block 214). The pathway provides an anchor for the catheter 402. The tunneling device may include a first end with a retractable sharp tip and a second end connected to a removable handle. After the tunneling device has created a desired pathway (usually before reaching the handle), the handle is removed and a second end of the catheter 402 is connected to a second end of the tunneling device.

A piercing device is then placed on an exterior of the skin tissue 308 at a desired location of an exit hole. The piercing device may include a needle that is surrounded by an alignment cup. The alignment cup is configured to accept the first end of the tunneling device through the skin tissue 308. A blunt tip of the tunneling device provides pressure on the skin to enable the needle of the piercing device to pierce the skin tissue 308 from outside the skin to create a catheter exit hole (block 216).

In some embodiments, the piercing device may be omitted and the piercing device instead uses the sharp tip to create the catheter exit hole from inside the skin tissue 308. The tunneling device is then pulled/pushed through catheter exit hole until the second end of the catheter 402 (connected to the second end of the tunneling device) extends or protrudes from the exit hole (block 218). The second end of the catheter 402 may then be connected to peritoneal dialysis tubing for a peritoneal dialysis treatment (block 420). The example procedure 200 of FIG. 2 then ends.

### Puncture Device Embodiments

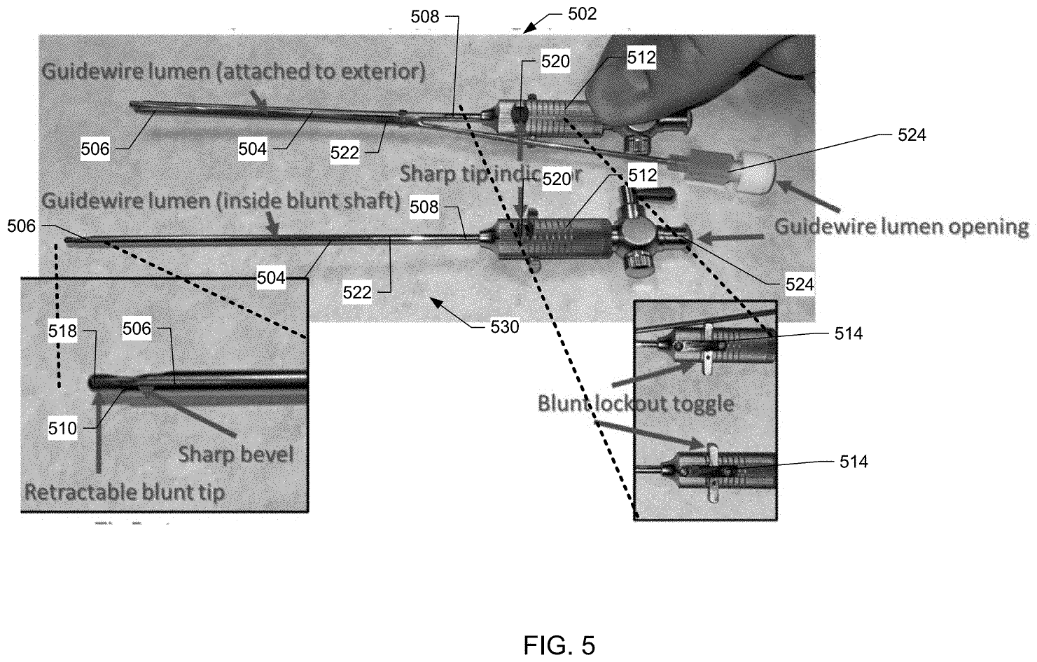

As discussed above in connection with FIGS. 2 to 4, the example catheter placement method 200 uses a puncture device. FIGS. 5 to 9 are diagrams of example puncture devices, according to example embodiments of the present disclosure. FIG. 5 shows a diagram of two modified veress safety needles that may be used as the puncture device 304 discussed in connection with FIG. 3. Each safety needle discussed herein includes a lumen for a guidewire, a lockable blunt tip, and an indicator regarding a status of a blunt/sharp tip.

A first puncture device 502 includes a shaft 504 having a first end 506 and a second end 508. The first end 506 includes a sharp bevel 510 configured for piercing through skin tissue, fat tissue, muscle tissue, and/or a peritoneal wall. The shaft 504 has a diameter between 2 and 15 mm and be formed of any material including stainless steel, metal alloys, plastic, composites, or combinations thereof,

The puncture device 502 includes a handle 512 that is connected to the second end 508 of the shaft 504. The handle 512 has a diameter that is greater than a diameter of the shaft 504. In some instances, the handle 512 includes a channel than enables the shaft 504 to pass through. The handle 512 includes an engagement lever 514 and/or a lockout toggle. The engagement lever 514 is connected to a blunt tip 518 via a retraction mechanism, which may include a rod connected to a spring and a holder.

The engagement lever 514 is movable between a sharp bevel exposure position to a blunt tip exposure position. In the sharp bevel exposure position, the holder of the engagement lever 514 or the retention mechanism enables the spring (e.g., the spring of FIG. 3) to compress. Compression of the spring from external tissue resistance causes the blunt tip 518 to be retracted into the shaft 504, thereby enabling the bevel 510 to be a leading edge. Movement or actuation of the engagement lever 514 from the sharp bevel exposure position to the blunt tip exposure position prevents the spring from retracting, thereby keeping the blunt tip 518 exposed when resistance from tissue is received. In some embodiments, the engagement lever 514 may include a dead man's switch where the blunt tip 518 may only be retractable when the lever 514 is actuated or depressed.

In alternative embodiments, a lockout toggle may be used instead of the engagement lever 514. When moved to the lock position, the lockout toggle prevents retraction of the blunt tip 518 when a force is applied, as shown at Event D in FIG. 3. When moved to the unlock position, the lockout toggle enables the blunt tip 518 to move to the sharp bevel exposure position and/or enables a force to cause the blunt tip 518 to be pushed into the shaft 504, thereby exposing the bevel 510.

The handle 512 of the puncture device 502 also includes an indicator 520. The example indicator 520 is configured to provide a visual indication as to whether the bevel 510 and/or the blunt tip 518 is positioned as the leading edge based on a position of the engagement lever 514. The visual indication may include a color (e.g., red to indicate the bevel 510 is the leading edge), a pattern, a symbol, etc. As shown in FIG. 5, the indicator 520 of the puncture device 502 shows a first indication that is indicative of the bevel 510 being the leading edge. For comparison, the indicator 520 of puncture device 530 shows a second indication that is indicative of the blunt tip 518 being the leading edge. Movement between positions of the engagement lever 514 may cause the indicator 520 to switch which visual indication is shown. In some embodiments, the indicator 520 may show a symbol or other visual indication when the engagement lever 514 is in the lock position. The indicator 520 informs a clinician as to whether the blunt tip 518 or the bevel 510 is the leading edge of the puncture device 502 even when the first end 506 is obscured from view by patient tissue.

The example puncture device 502 also includes a guidewire lumen 522 having a first end that is adjacent to the first end 506 of the shaft 504. The guidewire lumen 522 also includes a second end 524 that is offset from the handle 512. A lumen opening is attached to the second end 524 for receiving and directing a guidewire to the lumen 522. For the puncture device 502, the guidewire lumen is attached to an exterior surface of the shaft 504. Close to the handle 512, the guidewire lumen 524 splits from the shaft 504 at an acute angle.

The puncture device 530 of FIG. 5 shows another embodiment where the guidewire lumen 522 is located inside of the shaft 504 and/or the handle 512. The shaft 504 may include a first lumen for the blunt tip 518 and a second parallel lumen for the guidewire lumen 522. In some instances, the second lumen for the guidewire lumen 522 may be located within the first lumen or the blunt tip 518. The second end 524 of the guidewire lumen 522 is positioned at an end of the handle 520.

In some embodiments, the first end 506 of the puncture devices 502 and/or 530 may include an echogenic texture for ultrasound visualization. The echogenic texture provides an indication in an ultrasound as to a position of the first end 506. This indication helps a clinician locate the first end 506 relative to patient tissue to prevent an inadvertent cut or puncture.

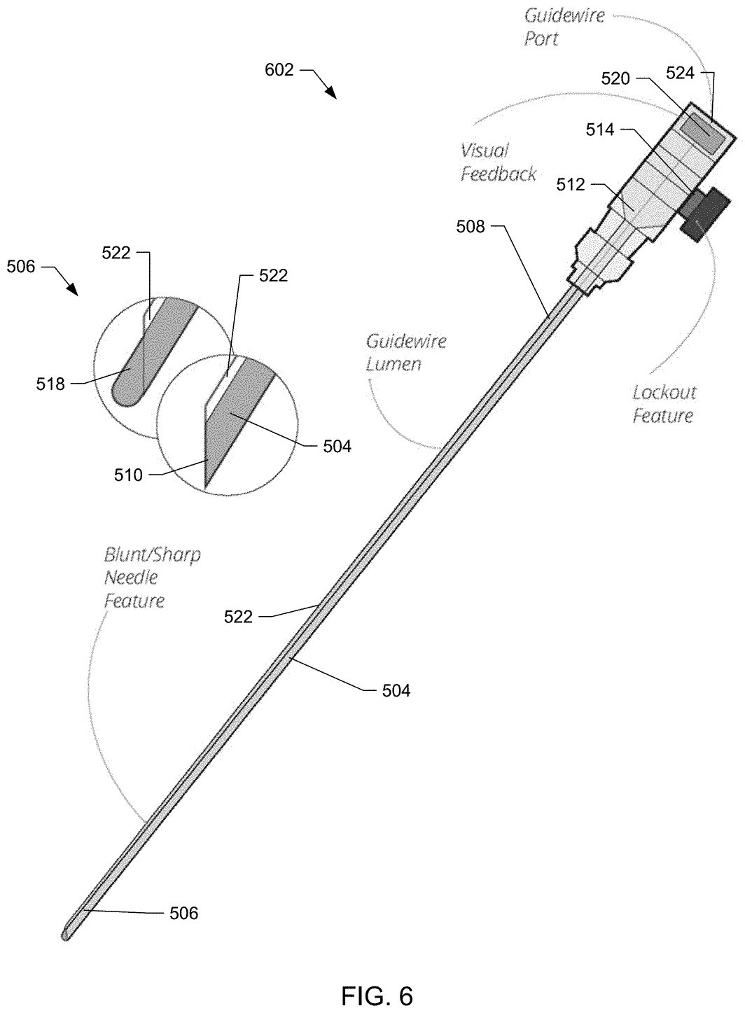

FIG. 6 is a diagram of another puncture device 602, according to an example embodiment of the present disclosure. The puncture device 602 is similar to the puncture device 530 of FIG. 5 and includes a shaft 504 having a first end 506 and a second end 508. The second end 508 of the shaft 504 is connected to a handle 512, which enables use of the engagement lever 514 and an indicator 520. In this embodiment, a guidewire lumen 522 is connected or integrally formed with the shaft 504, as shown in an enlargement of the first end 506 of the shaft 504. The guidewire lumen 522 is parallel to the shaft 504 through the handle 512, where a second end 524 is positioned with an opening to receive a guidewire. In some embodiments, the engagement lever 514 may be omitted such that a retractable blunt tip 518 may be retracted when force is externally applied.

The enlargement image of the first end 506 of the shaft 504 shows the two possible leading edges for the puncture device 602. In a first instance, the retractable blunt tip 518 is shown extending from the shaft 504 and past a bevel 510. In a second instance, the blunt tip 518 is retracted, thereby leaving the bevel 510 (integrally formed with the shaft 504) for piercing tissue. In some embodiments, the blunt tip 518 may include a blunt obturator (e.g., an inner shaft) and the bevel 510 includes a sharp cannula (e.g., an outer shaft).

In some embodiments, the engagement lever 514 is actuated by clicking a button (similar to a mechanism in a click-pen) and may be located on a side or at a top of the handle 512. The engagement lever 514 may include a sliding pin with a detent. When pushed in one direction (e.g., an unlock position), the engagement lever 514 permits motion of a blunt or sharp tip. In other embodiments, the engagement lever 514 is actuated by at least one of twisting or tightening the handle 512, twisting or tightening the shaft 504 (or outer shaft), and/or twisting and/or tightening a knob. In yet other embodiments, the engagement lever 514 includes a vertical switch (similar to a light switch) or is actuated by twisting the shaft 504 to interfere with an inner shaft (e.g., a minor axis of one shaft may interfere with a major axis of another shaft connected to the blunt tip 518). The engagement lever 514 may be actuated by pumping a pneumatic button or bulb, may use a magnet that when pushed into place attracts a magnet on one of the shafts, thereby preventing movement, actuated by rolling a dial or scroll wheel, or actuated on the handle 512 such that wings or bumps protrude when unlocked, and are hidden when locked. Further, the engagement lever 514 may be actuated by squeezing a flexible handle 512, actuated by squeezing a flexible handle 512 to remove the handle's interference with the shaft 504 (e.g., an oval cross section that deforms to allow movement), actuated by sliding a wedge into the shaft 504 to prevent movement, actuated by sliding a button downwards (e.g., similar to a multi-color pen), and/or may reset after one use and defaults back to a blunt setting. An action may be performed to “cock” the engagement lever 514 to be ready to actuate to the bevel again as a type of fail-safe. In some instances, the engagement lever 514 may be permanently locked until a clinician performs an action to activate movement (e.g., squeeze the handle 512, push a button, remove a pin, flip a switch, etc.). The engagement lever 514 defaults back to a locked position when not in use. (i.e., similar to a “deadman's switch” in valves or other mechanisms). The engagement lever 514 may be actuated after two “actions” to prevent accidental actuation (i.e., similar to a “double-click”). Moreover, the engagement lever 514 may enable rectus sheath tunneling with a needle during insertion.

In some embodiments, the indicator 520 may be angled to provide for a larger exposed color area. The indicator 520 may include a reflector, bright color and/or patterns, symbols, lines etc. to indicate when the bevel 510 is the leading edge. The indicator 520 may have a magnifying lens placed over it so that the lens magnifies a visual indication to make it appear larger and easier to see. The indicator 520 may include a diffusing lens to scatter the color to a greater viewing area. In some instances, the indicator 520 is visible from a top of the handle 512 such that the colors rotate into view as the tip 510/518 is actuated. The indicator 520 may also deliver other sensory feedback other than visual colors (e.g., audio or tactile feedback—clicks, vibrations, increased resistance, tactility/hardness of handle changes, etc.). The indicator 520 may become visible when an internal light path is either occluded or not occluded by the shaft 504 or the engagement lever 514. The indicator 520 may be integrated with the engagement lever 514. In some embodiments, the indicator 520 may change a physical shape of the handle 512 or the shaft 504 as the blunt tip 518 or the bevel 510 is actuated (e.g., protrusions are forced outwards when the bevel 510 is activated). The indicator 520 may protrude from a hole at the top of the handle 512 as a three-dimensional colored pin. In some instances, a hole for the indicator 520 is shrouded when not in use to prevent misidentifying the status of the tip 510/518 (e.g., red indicator tip emerges through rubber flaps or an opaque shroud and is only exposed when the bevel 510 is the leading edge). The indicator 510 may include a mirror to reflect the color from the side of the device 602 for easier viewing from above. Moreover, the indicator 520 may include an internal angled, circular mirror to reflect a color indicator on the shaft 504 upwards through a circular window on the handle 512.

In the illustrated example, the puncture device 602 enables a guidewire to be feed through the guidewire lumen 522. In some embodiments, the guidewire may include a fiber optic camera or a small borescope camera. The guidewire may vary between being still and flexible, where a flexible portion may be inserted into the peritoneum and a stiff portion is located in the shaft 504 of the puncture device 602. In some embodiments, the guidewire lumen 522 is replaced with a sheath pre-loaded on a needle. After accessing the peritoneum, the sheath is left in the patient while a guidewire is inserted. The sheath may be a standard or an expanding type.

It should be appreciated that the puncture device 602 (and/or the puncture devices 502 and 530) may be reversed such that the blunt tip 518 may be integrally formed with the shaft 504 and the bevel/sharp tip 510 may be retractable. In these examples, the bevel/sharp tip 510 may be retracted in a default position. In other embodiments, the puncture device 602 includes a dilator and/or a pre-loaded sheath, which may eliminate the need for a guidewire. In addition to above, the shaft 504 of the puncture device 602 may include depth numbering or markings. Further, the shaft 504 may be curved or be formed of a material that enables bending such as a plastic or reinforced rubber.

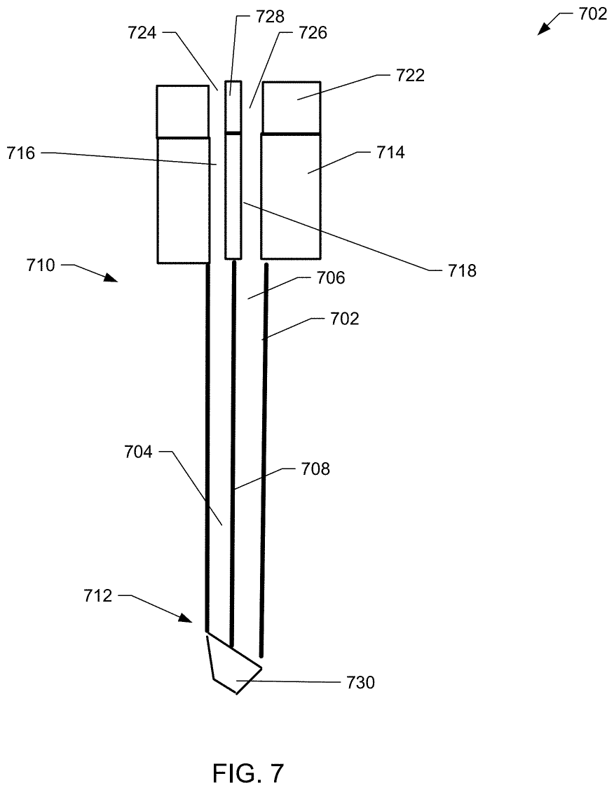

FIG. 7 is a diagram of another puncture device that includes a trocar 700, according to an example embodiment of the present disclosure. The trocar 700 includes a conduit 702 having a first lumen 704 and a parallel second lumen 706. In other examples, the conduit 702 may have three, four, or more parallel lumens.

The example conduit 702 may be formed using any rigid material such as, for example, stainless steel, aluminum, polypropylene, or any other surgical grade metal, plastic, or combinations thereof. The conduit 702 may also comprise glass, surgical grade steel, etc. The conduit 702 has a width between 3 millimeters (“mm”) and 15 mm and a length between 5 centimeters (“cm”) and 15 cm. A wall of the conduit may have a thickness between 0.1 mm and 0.8 mm.

The conduit 702 may include a single channel that is separated into two parallel halves (forming the lumens 704 and 706). The separation may be provided by a rigid structure or a flexible membrane 708. Alternatively, the conduit 702 may be formed as two parallel channels comprising the lumens 704 and 706. While only two lumens are shown, in other embodiments the conduit 702 may include three or more parallel lumens. Each of the lumens may have a same width or diameter or different widths/diameters based on which surgical instrument is to pass through. For example, a lumen designated for insufflation may have a smaller width/diameter compared to lumens for a catheter, an endoscope, and/or a laparoscopic manipulation tool.

The example conduit 702 includes a first end 710 and a second end 712. The first end 710 of the conduit 702 is located away from a patient while the second end 712 is configured for insertion into a patient to enable the trocar 700 to spread and hold incised tissue. In the illustrated example, the second end 712 includes a beveled edge for minimizing placement depth in a patient's peritoneum during an angled insertion. In other examples, the second end 712 includes a straight edge. The beveled edge or straight edge may include a blunt tip or a sharp tip to help incise skin. If the second end 712 includes a blunt tip, a scalpel may be used to first create an incision into the peritoneum. A veress needle or a guidewire may instead be used to create an initial puncture for the conduit 702 of the trocar 700.

As shown in FIG. 7, the first end 710 of the conduit 702 is connected to a head 714, which is configured to prevent the trocar 700 from slipping or otherwise moving completely through an incision into the patient. The head 714 has a width or a diameter that is greater than a width of a diameter of the conduit 702. For example, the width of the head 714 may be between 8 mm and 50 mm.

In the illustrated example, the head 714 includes a first lumen 716 and a parallel second lumen 718. The first lumen 716 is aligned with the lumen 704 of the conduit 702 and the second lumen 718 is aligned with the lumen 706 of the conduit 702. The alignment of the lumens 704, 706, 714 and 716 respectively provides for separation of surgical tools through the trocar 700.

In alternative embodiments, only the head 714 includes lumens 716 and 718. In these embodiments, the conduit 702 includes a single lumen. The dual lumens 716 and 718 in the head 714 provide sufficient separation of surgical tools such that multiple lumens in the conduit 702 are not needed. In yet alternative embodiments, the conduit 702 may include multiple lumens while the head 714 includes a single lumen or channel.

An example coupler 722 of FIG. 7 is connected to the head 714. In some embodiments, the coupler 722 may be integrally formed with the head 714. The example coupler 722 is configured to provide a secure connection to one or more surgical tools during a procedure. The secure connection prevents the surgical tools from unwanted movement during a procedure. The coupler 722 includes a first lumen 724 and a parallel second lumen 726 for receiving surgical tools. The lumens 724 and 726 are aligned respectively with the lumens 716 and 718 of the head 714 to enable surgical tools to pass through the trocar 700. The lumens 724 and 726 may be separated by a wall or partition 728 to prevent surgical tools from contacting each other.

The coupler 722 may also include a locking mechanism and/or a coupling mechanism to provide a pneumatic seal and/or to reduce a risk of contamination during a procedure. The locking/coupling mechanism of the coupler 722 may include a luer interface similar to blood and dialysis sets or locking threads. Alternatively, the locking/coupling mechanism of the coupler 722 may include a twist-to-lock mechanism with a pin and slot. The coupler 722 shown in FIG. 7 may include a gasket or other seal in conjunction with the locking/coupling mechanism. For example, a locking/coupling mechanism may be located at a top or mid-section of the coupler 722, while a gasket or seal is provided at a lower section of the coupler 722 closer to the head 714.

In some embodiments, the coupler 722 may include a side extension with a third lumen configured to accept an insufflation tool. The inclusion of a side extension enables the insufflation tool to be placed through the trocar 700 without interfering with other surgical tools that are placed through a top of the coupler 722. Further, the coupler 722 may be positioned between the head 714 and the conduit 702.

In addition to the embodiments above, the coupler 722 of FIG. 7 may include an on/off valve to permit an operator to control a gas or liquid into a peritoneal cavity of a patient. Alternatively, the coupler 722 may include a one-way input valve that enables gas or fluid to flow into the peritoneal cavity. In this alternative embodiment, the coupler 722 may include a separate vent valve to prevent hyper pneumoperitoneum.

The trocar 700 also includes a removable obturator 730. The example obturator 730 may be placed into the trocar 700 during initial insertion into a peritoneal cavity of a patient to prevent patient fluid/tissue from entering into the conduit 702. The obturator 730 may be placed through a lumen 724 or 726 of the coupler 722, and a respective lumen 716 or 718 of the head 714. A locking/coupling mechanism of the coupler 722, described above, may secure the obturator 730 in place. In some embodiments, the obturator 730 passes through one of the lumens 704 and 706 of the conduit 702 such that an end of the obturator extends from the conduit 702. To fully seal the conduit 702, the obturator 730 may press against the wall or membrane 708 of the conduit 702, thereby closing a second lumen.

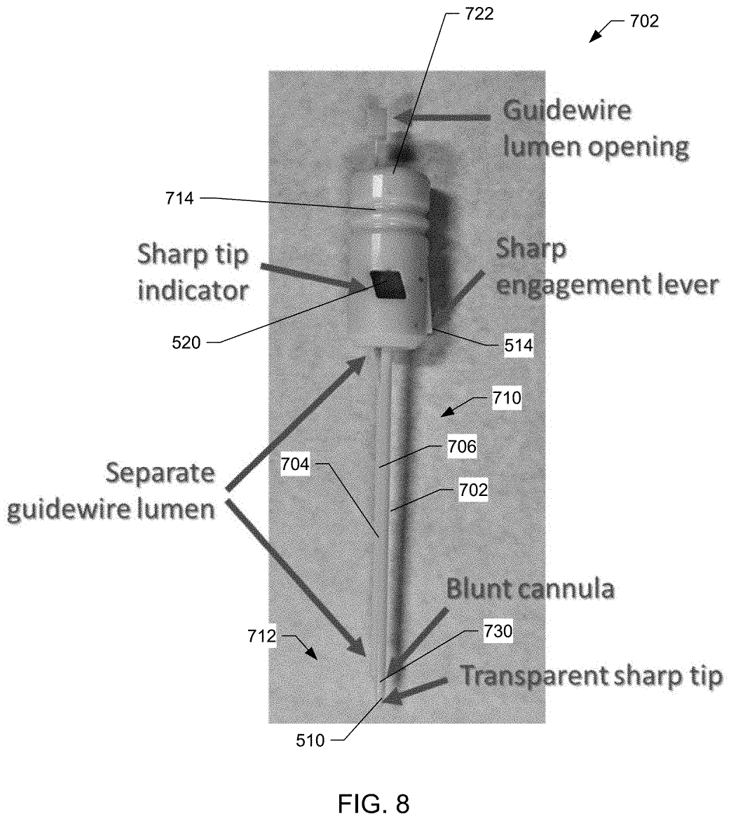

FIG. 8 is a diagram of the trocar 702 of FIG. 7 including an engagement lever 514, according to an example embodiment of the present disclosure. In this example, the engagement lever 514 enables the obturator 730 (e.g., a blunt tip or cannula 518) to extend or retract into the conduit 702 or shaft. The engagement lever 514 may include a dead man's switch in some embodiments. A bevel 510 or sharp tip is integrally formed into a second end 712 of the conduit 702. The engagement lever 514 may prevent the blunt tip or the obturator 730 from being retracted during use. The trocar 702 also includes an indicator 520 to provide an indication as to whether the obturator 730 or the sharp tip/bevel 510 is a leading edge.

FIG. 8 also shows that the first lumen 704 is a guidewire lumen and a second lumen 706 provides for retraction of the blunt tip/obturator 730. In some embodiments, the second lumen 706 may additionally or alternatively enable an endoscope (having a diameter that is less than 6 mm) to pass through. In these embodiments, the blunt tip or the obturator 730 may include a tube connected on an outside of a sharp inner portion 510 of the conduit 702. The sharp inner portion 510 may include a transparent viewing window, which enables visualization when an endoscope is placed in the second lumen 706.

A guidewire may be inserted into the trocar 702 via the coupler 722 and/or the head 714. A conduit within the head 714 directs the guidewire to the first lumen 704. The coupler 722 may include a locking/coupling mechanism for securing the guidewire and/or an endoscope in place.

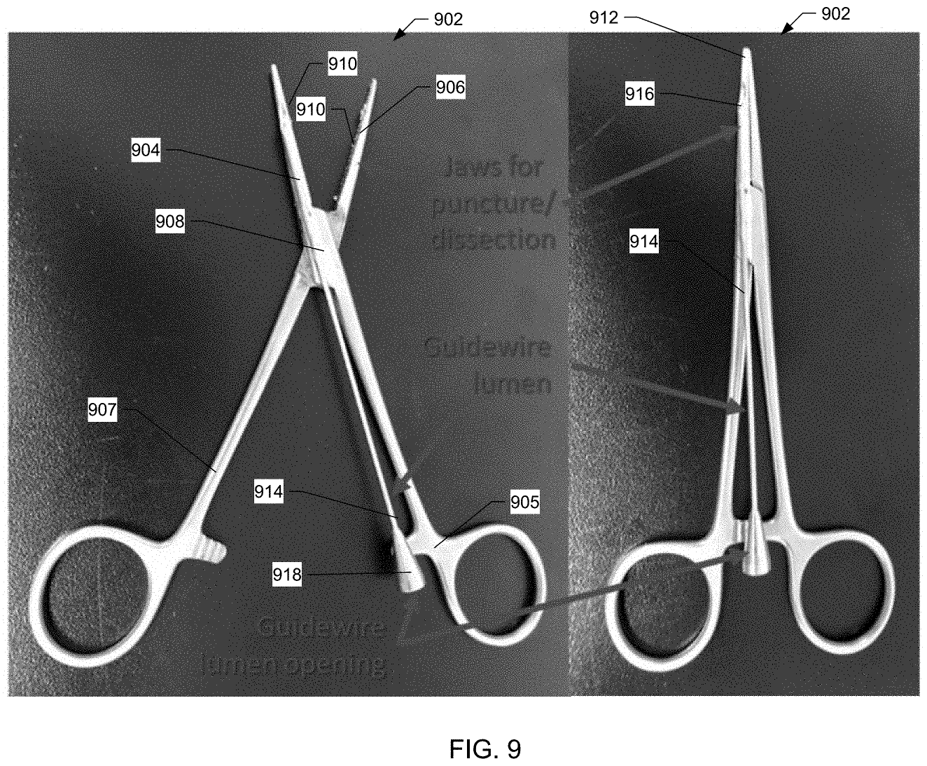

FIG. 9 is a diagram of a puncture device that includes a mosquito hemostat 902, according to an example embodiment of the present disclosure. In this example, the hemostat 902 includes a first jaw 904 connected to a first handle 905 and a second jaw 906 connected to a second handle 907, which pivot about a connection point 908. In this example, the jaws 904 and 906 include teeth 910 for holding tissue. Blunt outer edges of the jaws 904 and 906 provide for puncturing or piercing patient tissue similar to the sharp tip/bevel 510 of the puncture devices 502, 530, and 602. The blunt tip 912 may provide for safe puncturing of skin, fat tissue, muscle tissue, and the peritoneal wall, but blunt enough to prevent accidental cuts or scratches to internal tissue.

The example mosquito hemostat 902 includes a guidewire lumen 914 that is connected to the first handle 905 and the first jaw 904. The guidewire lumen 914 includes a first end 916 that is adjacent to the first jaw 904 to enable a guidewire to exit into a patient's peritoneal cavity. The guidewire lumen also includes a second end 918 for receiving the guidewire. The inclusion of the guidewire lumen 914 with the mosquito hemostat 902 enables the same tool to be used to puncture a patient and route a guidewire. Further, the jaws/handles of the mosquito hemostat 902 have a diameter that is smaller than a diameter of a catheter. This means a sheath or dilator may be needed for catheter placement after the mosquito hemostat 902 is removed.

### Endoscope Integration Embodiments

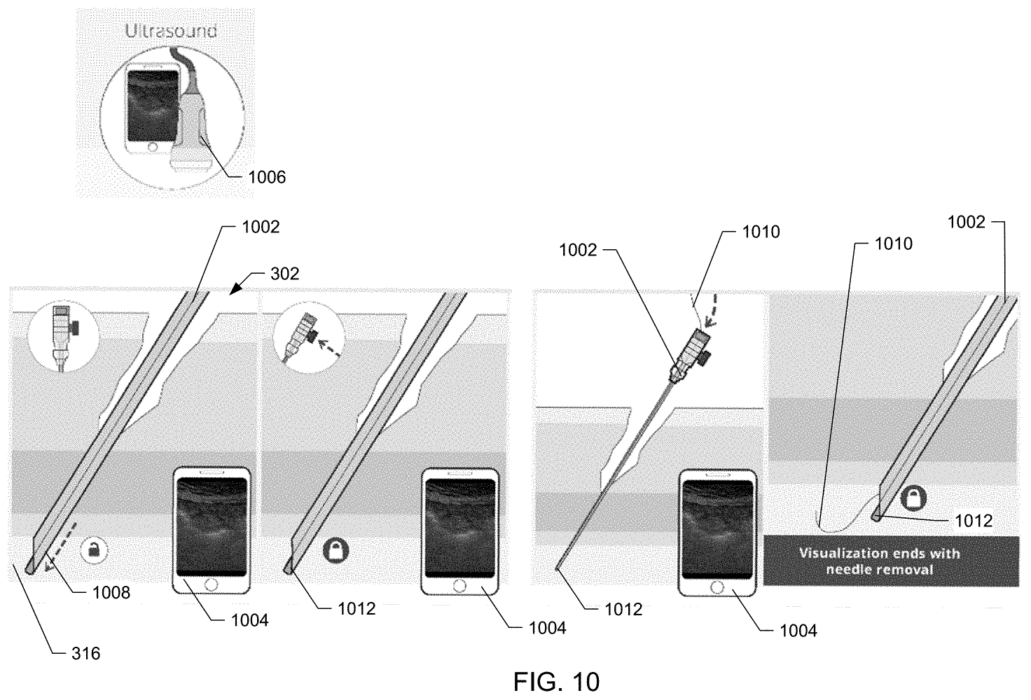

In some embodiments, the puncture devices discussed in connection with FIGS. 5 to 9 are configured to accommodate an endoscope or other visualization instrument. FIGS. 10 to 13 are diagrams of a puncture device operating in conjunction with an endoscope or other visualization instrument, according to example embodiments of the present disclosure. The integration of an endoscope or other visualization instrument provides an in-patient view for guidewire insertion. The visualization enables a clinician to see a patient's bowels and other internal tissue to avoid contacting with a puncture device and/or guidewire. The visualization may be used to confirm the puncture device is located fully inside the peritoneal cavity instead of being between pre-peritoneal and intraperitoneal walls. Visualization also enables observation of the peritoneal cavity for adhesions, hernias, or omentum interferences. Observation of these conditions are helpful during catheter insertion procedures to further assess patient health and provide additional information if complications occur after insertion (e.g. occlusions, omental wrapping, etc.).

FIG. 10 is a diagram showing visualization using ultrasound. In this example, a puncture device 1002 is used with a user device 1004 (e.g., a smartphone, a tablet computer, a laptop computer, a server, a workstation, etc.) and an ultrasound device 1006. The puncture device 1002 may be similar to the puncture devices 304, 502, 530, 602, 702, and 902 discussed in connection with FIGS. 3 and 5 to 9. An end 1008 of the puncture device 1002 includes echogenic texture for ultrasound visualization.

As shown in FIG. 10, the end 1008 of the puncture device 1002 creates an access hole 302 using a bevel. During insertion of the puncture device 1002 into the peritoneal cavity 316, the ultrasound device 1006 is placed outside the skin. The ultrasound device 1006 is communicatively coupled to the user device 1004 via a wired or wireless connection. An application on the user device 1004 renders and displays data from the ultrasound device 1006, showing a visualization of the patient's peritoneal cavity 316. The echogenic texture is readily viewable in the display on the user device 1004, which enables a clinician to see where the end 1008 of the puncture device 1002 is located relative to patient tissue. In some embodiments, the ultrasound device 1006 may be replaced by a fluoroscopy machine.

After the puncture device 1002 is placed at a desired location or at a desired depth within the peritoneal cavity 316, a clinician routes a guidewire 1010 through a guidewire lumen of the puncture device 1002. During this time, a blunt tip 1012 is placed into a locked position to prevent accidental piercing by the bevel of the puncture device 1002. The guidewire 1010 exits the first end 1008 and is routed to an appropriate location. After insertion of the guidewire 1010, the puncture device 1002 is removed, leaving the guidewire in place. A catheter may then be inserted via the guidewire 1010.

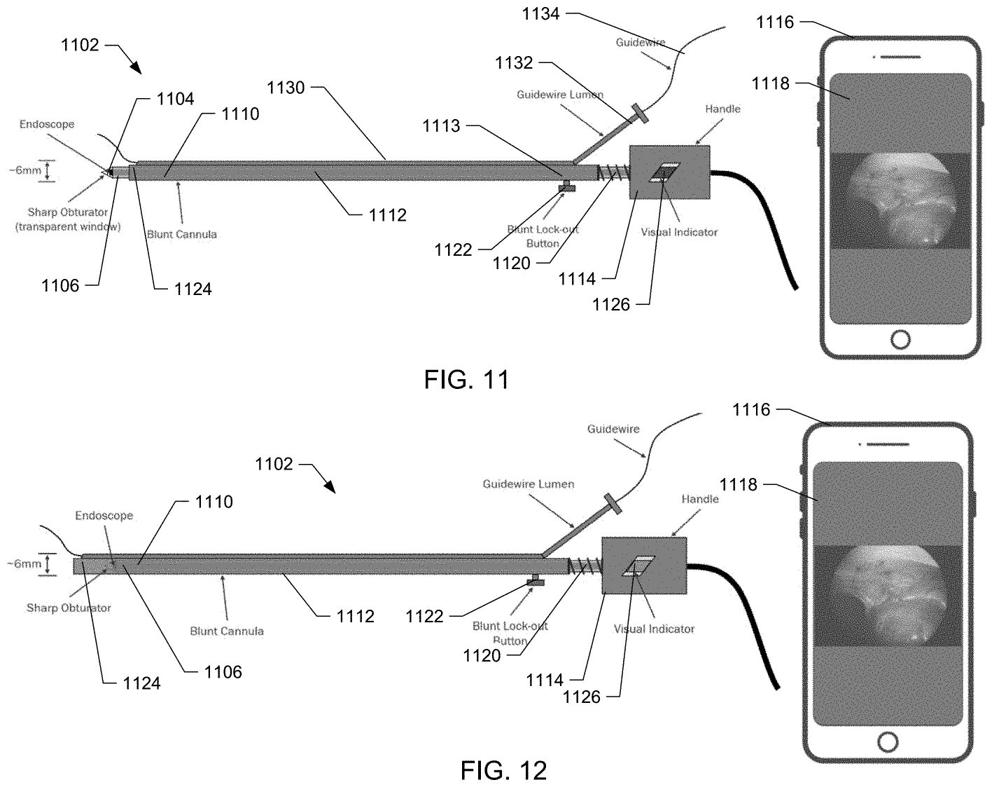

FIGS. 11 and 12 are diagrams of a puncture device 1102 including an endoscope 1104, according to example embodiments of the present disclosure. In this example, the puncture device 1102 includes a retractable sharp tip 1106. At least a portion of the retractable sharp tip 1106 includes a transparent material. The endoscope 1104 is positioned within the sharp tip 1106 to provide visualization from a first end 1110 of the puncture device 1102.

The example endoscope 1104 includes any CCD/CMOS camera having a diameter (e.g., 2 to 4 mm) that is less than a diameter of the sharp tip 1106 and/or a shaft 1112 of the puncture device 1102. In some embodiments, the endoscope 1104 may include one or more LEDs to provide illumination. Alternatively, the endoscope 1104 may include a lens stack that projects an image from the sharp tip 1106 to a larger CCD/CMOS camera located at (or past) a handle 1114.

In the illustrated example, the endoscope 1104 is connected to a user device 1116 via a wired connection (e.g., a USB, HDMI, etc.) or a wireless connection (e.g., Bluetooth®, Zigbee®, etc.). In other embodiments, the wired connection may include a fiber optic connection to a transceiver, which is communicatively coupled to the user device 1116 via a wired or wireless connection. The example user device 1116 includes an application 1118 for displaying video images recorded by the endoscope 1104.

The example puncture device 1102 is similar to the puncture devices 304, 502, 530, 602, and 702 discussed in connection with FIGS. 3 and 5 to 8. The puncture device 1102 includes an engagement lever 1120 at a second end 1113 for retracting/extending the sharp tip 1106. The puncture device 1102 also includes a lockout toggle 1122 for preventing the sharp tip 1104 from extending past a blunt cannula or tip 1124. FIG. 12 shows the sharp tip 1106 in a retracted position, thereby enabling the blunt tip 1124 to be the leading edge of the puncture device 1102. The sharp tip 1106 is retracted into the first end 1110 of the shaft 1112. In this embodiment, the sharp tip 1106 may include a retractable obturator and the blunt tip 1124 includes a blunt outer end of the shaft 1112. As described in FIGS. 3, 5, and 6, the blunt tip 1124 may be reversed with the sharp tip 1106 such that the sharp tip is integrally formed with the shaft 1112 and the blunt tip 1124 is retractable.

The example puncture device 1102 also includes an indicator 1126 located on the handle 1114. In FIG. 11, the indicator 1126 shows a first color or pattern to indicate the sharp tip 1106 is extended past the blunt tip 1124. In FIG. 12, the indicator 1126 shows a second color or pattern to indicate the sharp tip 1106 is retracted and the blunt tip 1124 forms a leading edge.

The example puncture device 1102 also includes a guidewire lumen 1130 that is connected to the shaft 1112. A combined diameter of the shaft 1112 and the guidewire lumen 1130 is about, for example, 6 to 8 mm. A first end of the guidewire lumen 1130 is located at the first end 1110 of the shaft and is offset or set back from the blunt tip 1124. A second end 1132 of the guidewire lumen 1130 connects to the shaft 1112 at an angle. The second end 1132 includes an opening for receiving a guidewire 1134.

In some embodiments, the puncture device 1102 of FIGS. 11 and 12 may include a built-in dilator and a pre-loaded sheath. The dilator and sheath may eliminate the need for a guidewire. Further, the shaft 1112 of the puncture device 1102 may include depth numbering or markings on an exterior surface. Moreover, in some embodiments the shaft 1112 may include a third lumen for the endoscope 1104 such that integration with the sharp tip 1106 is not needed. This separate shaft enables the endoscope 1104 to be extended past the first end 1110 of the shaft. In yet other embodiments, the guidewire 1134 may include a fiber optic camera or a small borescope camera.

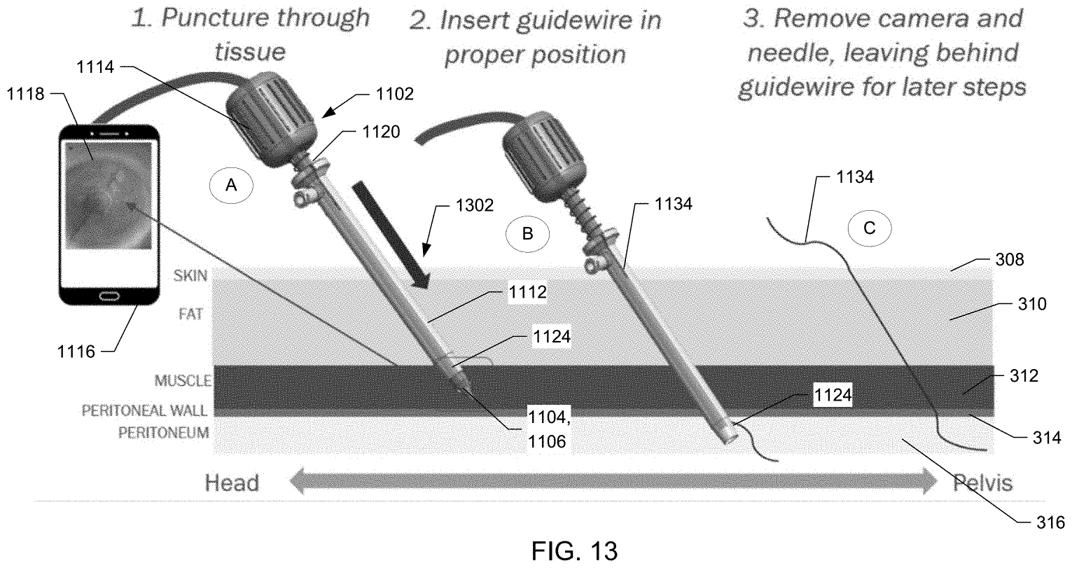

FIG. 13 shows a method of using the puncture device 1102 of FIGS. 11 and 12, according to an example embodiment of the present disclosure. At Event A, the sharp tip 1106 and the endoscope 1104 are in an extended position. To provide the extension, the engagement lever 1120 (including the shaft 1112) is moved toward the handle 1114, which causes the sharp tip 1106 and the endoscope 1104 to be pushed from the shaft 1112. In this position, the puncture device 1102 pierces through fat tissue 310, and/or muscle tissue 312 to create an access hole 1302 (where the skin tissue 308 may be dissected via a scalpel).

At Event B, the puncture device 1102 pierces through the peritoneal wall 314 and reaches the peritoneal cavity 316. At this point, the engagement lever 1120 is released, which enables a spring to extend to an uncompressed state, thereby pushing the shaft 1112 downward. This downward movement causes the sharp tip 1106 to be retracted into the shaft 1112, which enables the blunt tip 1124 to be the leading edge. The endoscope 1106 may continue to provide visualization through an opening of the shaft 1112 at the blunt tip 1124. Also at Event B, a guidewire 1134 is routed through a guidewire lumen of the puncture device 1102 and exits into the peritoneal cavity 316.

At Event C, after the guidewire 1134 is placed into a desired location, the puncture device 1102 is removed. A catheter may then be inserted along the guidewire 1134 through the access hole 1302. Since a diameter of the puncture device 1102 is approximately equal or similar to a diameter of the catheter, the diameter of the access hole 1302 does not have to be further enlarged, thereby reducing a number of steps for catheter insertion.

### Tunneling Device Embodiment

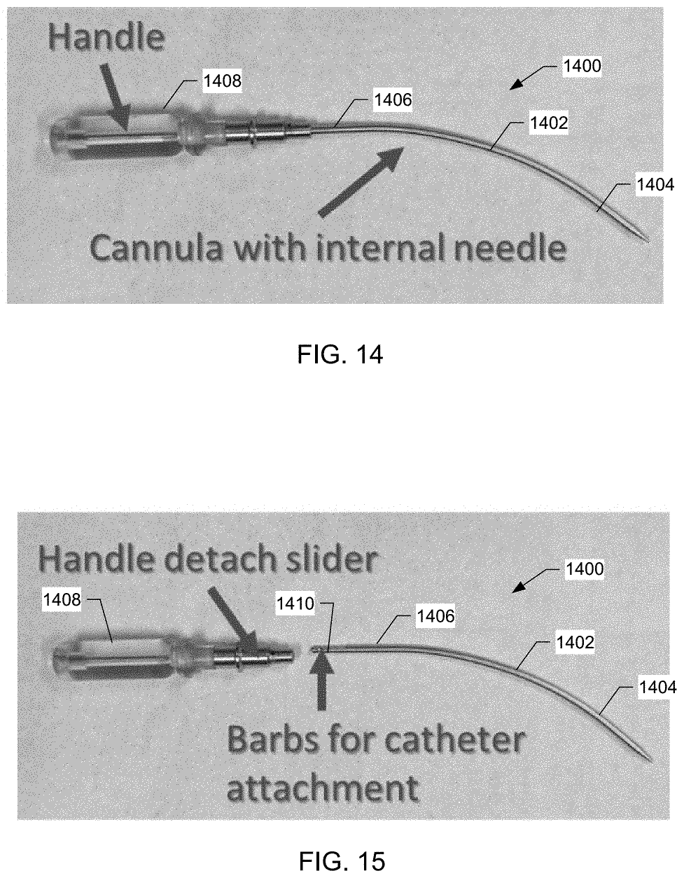

As described above in connection with FIG. 2, a tunneling device is used to create a pathway through fat tissue to anchor a catheter. FIGS. 14 to 17B are diagrams of a tunneling device 1400, according to an example embodiment of the present disclosure. The tunneling device 1400 includes a shaft or cannula 1402 with a retractable needle. The shaft 1402 includes a first end 1404 with the retractable needle for creating a pathway through fat tissue. The shaft 1402 also includes a second end 1406 that is releasably connected to a handle 1408. The shaft 1402 may have a diameter between 2 mm and 15 mm, for example, and be formed from stainless steel, a metal alloy, plastic, rubber, or combinations thereof.

As shown in FIG. 14, the handle 1408 is connected to the second end 1406 of the shaft 1402. In FIG. 15, the handle 1408 is shown detached from the second end 1406 of the shaft 1402. The handle 1408 may be detachable by a sliding mechanism (e.g., a ball and detent) such as used with a pneumatic connector. The second end 1406 may include one or more barbs 1410 to enable attachment to a catheter. In other embodiments, the second end 1406 may include other connectors to connect to an end of a catheter.

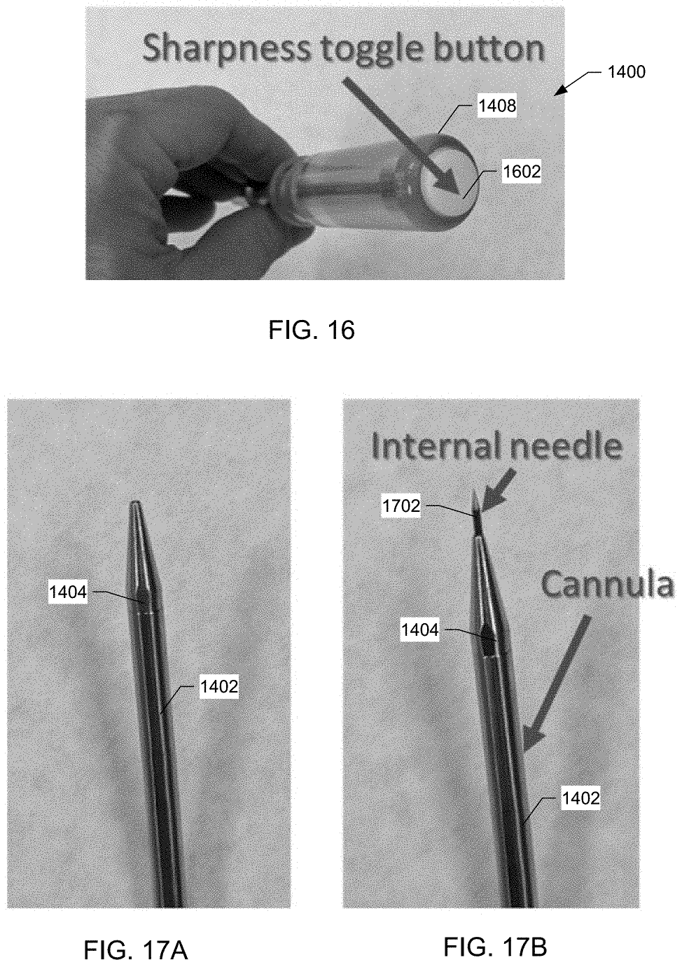

FIG. 16 is a diagram that shows a toggle button 1602 on the handle 1408. Actuation of the button 1602 causes a needle 1702 (shown in FIG. 17B) to extend from the first end 1404 of the shaft 1402. The button 1602 may be released, causing the needle 1702 to retract into the first end 1404 of the shaft 1402. When the handle 1408 is detached from the shaft 1402, the needle 1702 retracts into the shaft 1402. The example needle 1702 is used for piercing through skin. In some embodiments, the first end 1404 of the shaft 1402 may be removable via a threaded connection to provide access for cleaning.

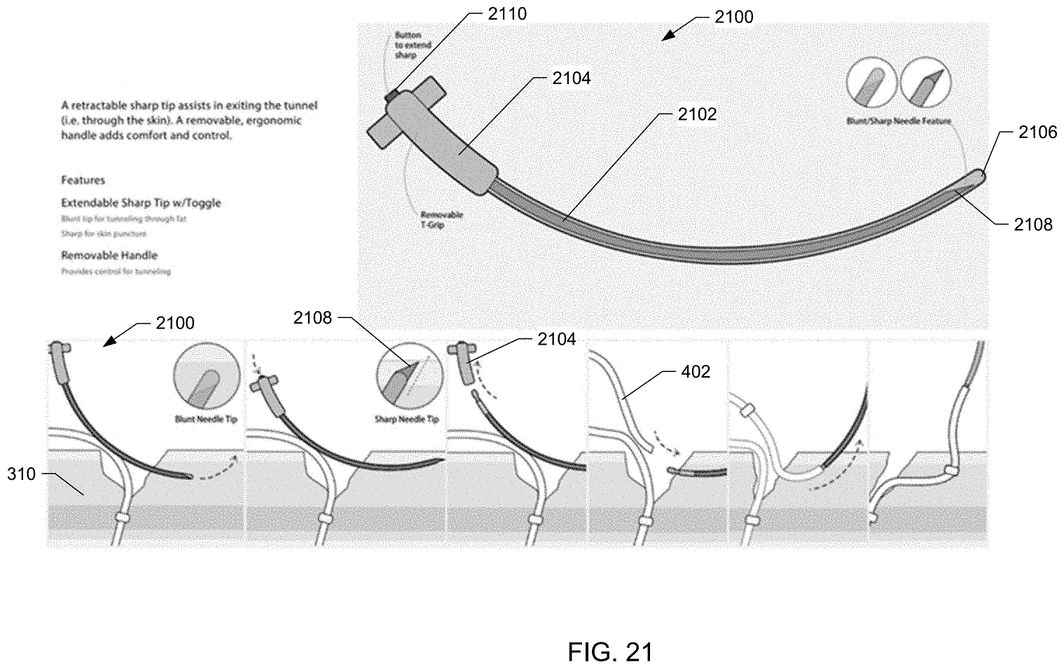

FIGS. 21 to 27 are diagrams of another embodiment of a tunneling device 2100, according to an example embodiment of the present disclosure. In the illustrated embodiment, the tunneling device 2100 includes a shaft 2102 that is removably connected to a handle 2104 at a first end. A second end of the shaft 2102 includes a blunt tip 2106 and a retractable sharp tip 2108. A button 2110 on the handle 2110 may be depressed or otherwise actuated to cause the sharp tip 2108 to extend past the blunt tip 2106.

As shown in the diagram sequence, the tunneling device 2100 is inserted into a fat tissue layer 310 with the sharp tip 2108 retracted, leaving the blunt tip exposed 2106. When the tunneling device 2100 is routed to a desired catheter exit hole, a user engages the button 2110, causing the sharp tip 2108 to extend past the blunt tip 2106. A user then pierces through the patient's skin layer using the sharp tip 2108. After the catheter exit hole has been formed, the handle 2104 is removed. A catheter 402 is then connected to the first end of the shaft 2102 in place of the handle. The tunneling device 2100 is then pulled through the hole in the patient's skin, thereby pulling the catheter 402 through the fat layer and also out through the exit hole. At this point, the tunneling device 2100 may be detached from the catheter 402 and a skin sealant may be applied to prevent blood/fluid leakage.

The example tunneling device 2100 is unique over known tunneling devices by proving a removable handle 2104 and a sharpness toggle. The tunneling device 2100 provides safety and efficacy for a tunneling step of a catheter insertion procedure. In the case of PD catheter implantation, the tunneling device 2100 may reduce the risk of organ/vessel perforation or damage during a routing step. Additionally, the ability to directly puncture through the skin from the inside minimizes the risk of a misplaced or overly large skin puncture that may lead to complications later such as infection or post-operative fluid leakage. The act of tunneling and puncturing through the skin is also made ergonomically easier, requiring less strength or exertion. Lastly, the tunneling device 2100 may offer time savings by removing the need to puncture the skin externally with a separate scalpel/device to permit exit of the device through the skin.

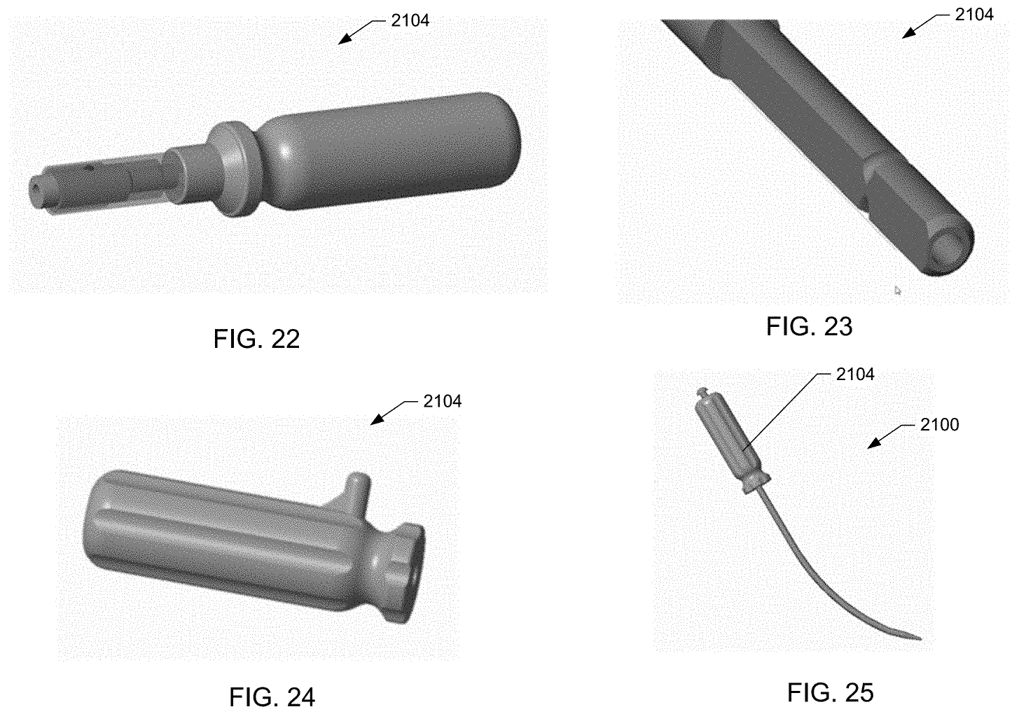

FIGS. 22 to 27 are diagrams showing alternative embodiments of the handle 2104. The example handle 2104 may have an ergonomic shape to provide better leverage for a user. As shown in FIGS. 22 and 23, the handle 2104 may connect to the shaft 2102 of the tunneling device 2100 via a keyed or shaped connection to prevent incorrect rotation relative to a tunneling portion. For example, a square or hexagon shape may be used to enable several distinct rotation angles depending on user discretion. A slot of flat on the handle 2104 or shaft 2102 could also serve the same purpose. This could also be used to index several positions.

In some embodiments, the handle 2104 attaches to the shaft 2102 via magnets. The handle 2104 may alternatively attach to the shaft 2102 via a compression thread, similar to a tool “chuck.” Further, the handle 2104 may attach to the shaft 2012 via a spring-loaded pin that fits into a mating hole. The handle 2104 may also attach to the shaft 2102 via a ball and detent coupling, similar to ratchet sets and other common tools. As shown in FIGS. 22 and 23, the detent for the ball connection on the handle 2104 can also serve as a “barb” for attaching the catheter to ensure it does not slide off during tunneling. Additional barbs could be incorporated for a more robust connection.

The handle 2104 may also attach to the shaft 2102 via a screwing or twisting mechanism. This screwing also extends the sharp portion for the toggle as the button 2110. The handle 2104 may also attach to the shaft 2102 via compression of an elastomeric material by the user, instead of rigidly attaching to the tool. The handle 2104 may also attach to the shaft 2102 via a ratcheting mechanism that tightens the handle on to the shaft. The handle 2104 main axis could be in-line with the shaft 2102 main axis. Alternatively, the handle 2104 main axis could be perpendicular to the shaft 2102 main axis. This could resemble a “T,” similar to a thread tap or some Allen keys. The handle 2104 may include features to improve grip, such as flats, gnurling, protrusions, helpful angles, or other types of texturing. An example is depicted in FIG. 24.

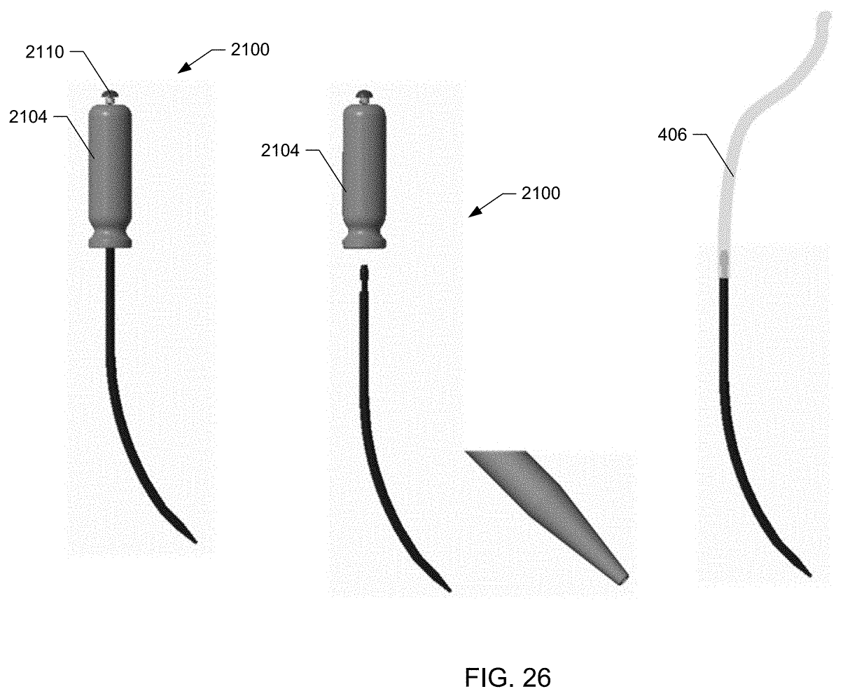

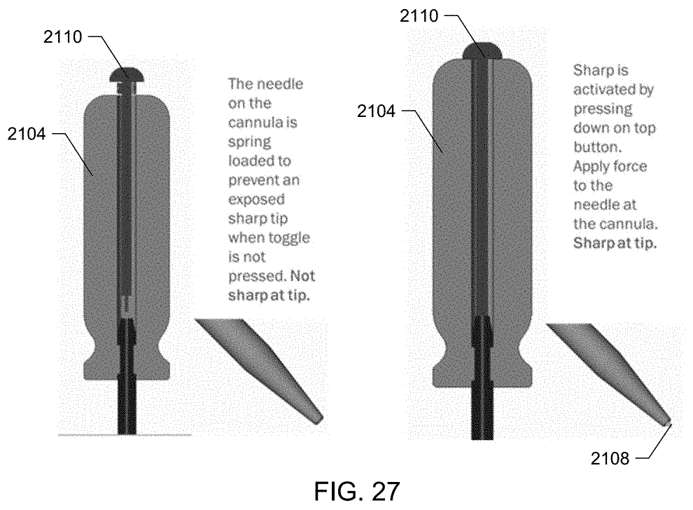

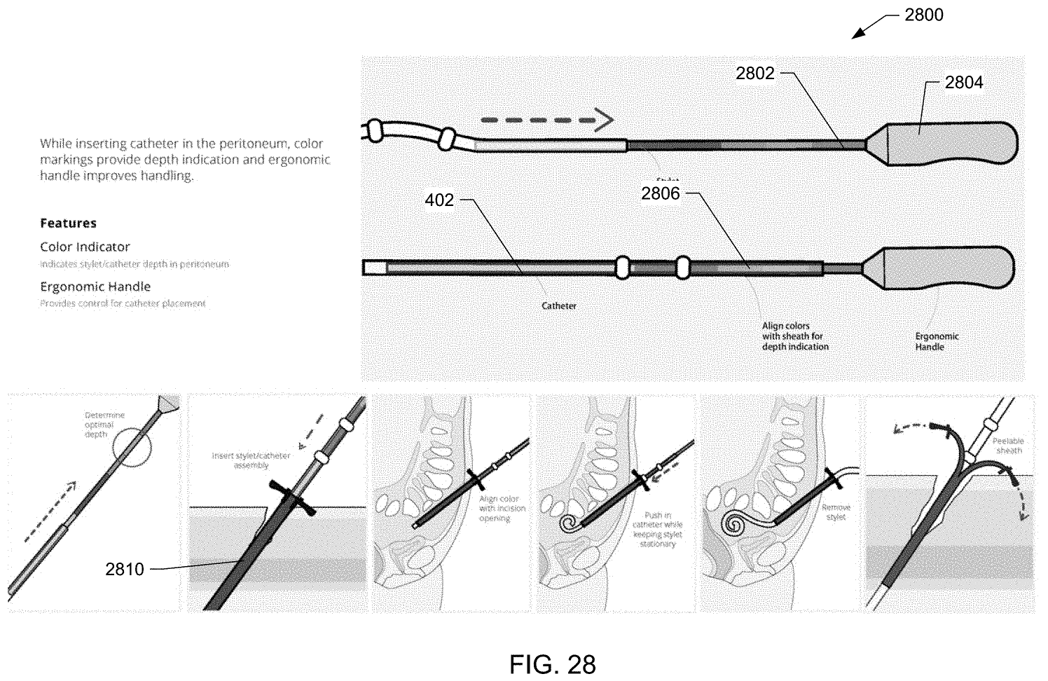

FIGS. 26 and 27 are diagrams that show sharp tip actuation of the tunneling device 2100 of FIGS. 22 to 25. The button 2110 may include a slider, a lever, a trigger, etc. that adjusts the sharpness of the shaft 2102. The button 2110 may include a fail-safe two-step mechanism that involves pushing in and then laterally moving or rotating to render the sharp tip 2108. The button 2110 may include a safety toggle like a firearm that needs to be moved/switched prior to pushing a button or trigger to toggle the sharp tip 2108. The button 2110 may also include a finger loop to slide the inner sharp shaft out (inside the blunt outer tool). The button 2110 may also include a trigger button like a firearm on the side . . . depress to activate. The button 2110 may also include a button that is covered by a hinged or sliding piece. The button 2110 may further include a slider or light switch-like mechanism that slides the inner sharp shaft within the outer blunt piece. In other embodiments, the sharp tip 2108 becomes sharp via an automatic, spring-loaded mechanism that is dependent on tissue resistance. The button 2110 may also include a twist mechanism that causes the sharp tip 2108 to extend or retract.

The tunneling device 2100 may include a sharpness mechanism for toggling the sharp tip 2108. The sharp tip 2108 may include a blunt tip with sharp edges like a drill bit, which requires twisting to puncture through the skin. The sharp tip 2108 may also include a sharp shaft within a dull outer cover that is spring loaded, and extends to become sharp upon user actuation. The sharp tip 2108 may further include a bistable mechanism: a single piece design that involves squeezing to change the tool geometry and rendering the end sharp. The sharp tip 2108 may also include a septum made of rubber or other material that reduces likelihood of tissue debris getting in between the middle needle obturator and an outer portion.

When disassembling during a procedure, the shaft 2102 may include a safety mechanism that automatically covers the sharp tip 2108 once it is removed from the outer trocar. This mechanism could be incorporated as a semi-compressible rubber piece that extends when the handle 2110 and the sharp tip 2108 are removed, but is partially or fully compressed during the procedure. This mechanism could also function as a spring for sharpness actuation.

The sharp tip 2108 may also include a 2-piece sharp mechanism: a blunt shaft linked to the handle 2110 that pushes against a separate sharp segment ending in the tip of the shaft 2102. A variety of length ratios could be utilized for the sharp and dull segments (e.g., short sharp segment near tip of the shaft 2102 and/or a much longer blunt segment attached to handle 2110). The purpose of a 2-piece design is ease in safe disassembly during a procedure and later possible reprocessing. An example is depicted in FIG. 27.

The shaft 2102 may include a feature for tunneler-catheter orientation for aligning a catheter to the tunneling device 2100 to reduce the risk of catheter kinks or twists. This can include a rotating collar on the handle 2110 that has a mark for where a radioopaque strip should align. Several stripes on the tunneling device 2100 may indicate where the catheter radioopaque strip should align. A stripe may be placed on each side to accommodate different catheters and Left/Right handed users. The shaft 2102 may include flats or indents to indicate where the catheter radioopaque strip should align.

### Piercing Tool Embodiment

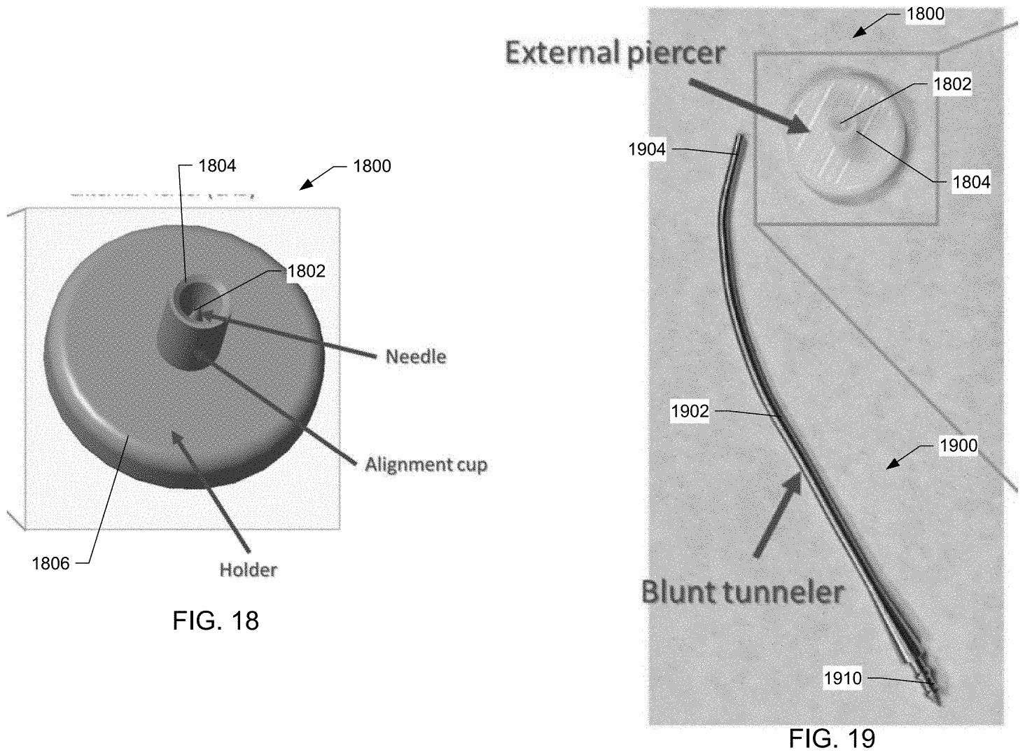

As described above in connection with FIG. 2, a piercing tool is used to help create a catheter exit hole. FIG. 18 is a diagram of a piercing tool 1800, according to an example embodiment of the present disclosure. The piercing tool 1800 includes a needle 1802 that is surrounded by an alignment cup 1804. The needle 1802 may be recessed within the alignment cup 1804 or extend from the cup. The example alignment cup 1804 is supported by a holder or base 1806. While the base 1806 is shown as being circular, in other embodiments the base may have a rectangular shape. The piercing tool 1800 may include stainless steel, a metal alloy, plastic, or combinations thereof.

FIG. 19 is a diagram that shows how the piercing tool 1800 of FIG. 18 is used with a blunt tunneling device 1900 to create a catheter exit hole, according to an example embodiment of the present disclosure. In the illustrated example, a tunneling device 1902 is located under a patient's skin tissue within a pathway of fat tissue. A blunt first end 1904 of the shaft 1902 is positioned on an interior side of a patient's skin where the exit hole is desired. The piercing tool 1800 is placed on an opposite, exterior side of the patient's skin at the desired exit hole. The alignment cup 1804 defines an opening that receives the first end 1904 of the shaft 1902. Pressure from the shaft 1902 causes the patient's skin to tent. The alignment cup 1804 is placed over the tented skin to provide alignment with the shaft 1902. This enables the alignment cup 1804 and the first end 1904 of the shaft 1902 to sandwich a portion of the patient's skin tissue at the exit hole. After alignment, the shaft 1902 is pushed toward the piercing device 1800 while at the same time the piercing device 1800 is pushed toward the first end 1904 of the shaft 1902. This causes the needle 1802 of the piercing tool 1800 to pierce the skin externally, reducing the chance of accidently puncturing a clinician and while creating a smaller, cleaner exit hole. In some embodiments, the needle 1802 may be spring loaded to aid in piercing. After the exit hole is formed, a catheter (connected to the barbs 1910) may be pulled through the pathway. The shaft 1902 of the tunneling device 1900 may be pulled all the way through the exit hole until an end of the catheter passes through. The catheter is disconnected from the barbs 1910, and may later be connected to PD tubing for a peritoneal dialysis treatment.

In some embodiments, a handle may be removed from the piercing device 1900 before the exit hole is formed. In these embodiments, the blunt tip of the first end 1904 aligns with the needle 1802 via the alignment cup 1804. The needle 1802 may then pierce through the skin to form the exit hole while the blunt tip of the shaft 1902 provides support from under the skin. When the needle 1802 pierces the skin, the alignment cup 1804 accepts the first end 1904 of the shaft 1902 to provide controlled piercing.

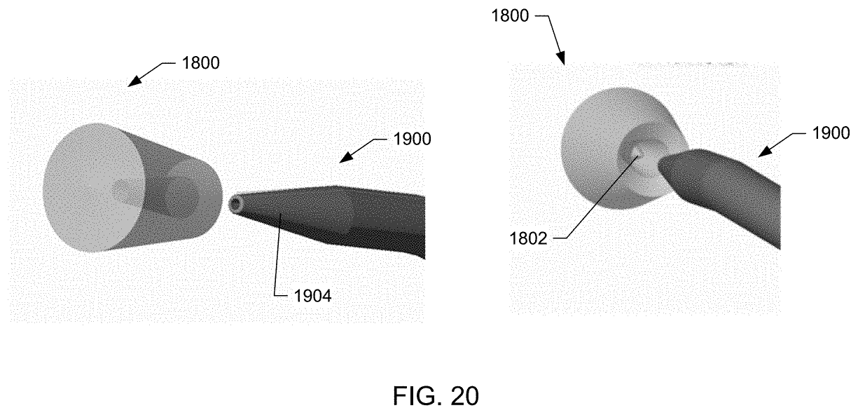

FIG. 20 is another diagram showing the blunt tunneling device 1900 and the piercing tool 1800, according to an example embodiment of the present disclosure. The piercing tool 1800 has a cylindrical shape that narrows from a base to a top end. A middle of the piercing tool 1800 is recessed, and gradually decreases in diameter towards the base. The needle 1802 is located in a center of the recessed section. The recesses section is configured to receive and direct the blunt tip of the first end 1904 towards the needle 1802, thereby piercing through a portion of a patient's skin layer.

### Guiding Stylet Embodiment

Peritoneal dialysis catheter placement sometimes involves the use of a rigid stylet. When used, the catheter is first loaded by sliding the catheter over the stylet. The stylet is then inserted into the access point into the peritoneum that had been prepared in a previous procedural step, as discussed above. This access point is typically either a peel-away sheath or expandable sheath, but in some cases may simply be dissected tissue. There are a wide variety of surgical stylets commercially available. However, known stylets are typically comprised of a thick wire with a bent looped end for a handle.

The disclosed stylet includes features to simplify or improve the catheter insertion process. Some of the improvements include “graduation” of the stylet, meaning there are color, texture, and/or numerical indicators to guide the inserter to the appropriate depth. Additionally, the stylet may include an ergonomic handle that permits easier manipulation of the tool during insertion.

FIG. 28 is a diagram of a guiding stylet 2800, according to an example embodiment of the present disclosure. The stylet 2800 includes a rigid shaft 2802, an ergonomic handle 2804, and one or more colors or markings 2806 to indicate a depth of insertion or when to stop insertion. As shown in FIG. 28, the colors or markings 2806 enable a user to determine an optimal depth. When a catheter 402 and connected stylet 2800 are inserted into a sheath 2810, the colors or markings 2806 are used to provide a depth indication, where a desired marking is aligned with an incision opening to ensure the catheter is provided at a desired depth. The catheter may then be pushed through the sheath 2810, while keeping the stylet 2800 stationary. The sheath 2810 may then be peeled away and the stylet 2800 may be removed from the catheter, thereby enabling the end of the catheter to be tunneled for anchoring to a patient.

In some instances, the stylet 2800 comes pre-loaded with a catheter. The stylet 2800 may include features to make it more ultrasound opaque (echogenic). The stylet 2800 may have variable stiffness along the length (e.g., a semi-flexible tip made of rubber, plastic or a coated spring). The stylet 2800 may include cuff-pushing features. The stylet 2800 may have a tapered dilator and cuff pusher built into it as an all-in-one tool. The stylet 2800 may include an extendable portion for larger or taller patients.

The stylet 2800 may include features or a mechanism to “grab” the catheter during insertion and then “release” the catheter when the stylet is ready to be removed. It would also enable easy loading of the catheter on the stylet. The stylet 2800 may include a clip to hold the catheter in place until ready to remove the catheter. The stylet 2800 may include rings, bumps, barbs, dimples, or a taper to hold the catheter on the stylet until ready to remove the catheter. The stylet 2800 may include a metal rod coated in plastic (e.g. Teflon™) or another material for friction reduction. The stylet 2800 may include a metal or rubber pad on the plastic handle for increased tactile feedback. The stylet 2800 may include a round/blunt tip which will not damage the catheter or tissue while being inserted.

The stylet 2800 may include colors as a depth indicator marker. The stylet 2800 may include symbols as a depth indicator marker. The stylet 2800 may include a pattern, dots, or lines as a depth indicator marker. The stylet 2800 may include numbers or numerals as a depth indicator marker. The stylet 2800 may include LED(s) to indicate depth. The stylet 2800 may include markings or color bands to show the correct placement of a deep cuff. The stylet 2800 may further include generic markings based on height-to-weight ratios or BMIs. The stylet 2800 may include multiple markings that can then be adjusted or moved into view based on the patient size. The stylet 2800 may include markings that are consistent among pieces of a catheter insertion kit (i.e., needle, sheath, catheter, etc. all have the same markings as the stylet). The stylet 2800 may include any combination of the above marking methods.

The stylet 2800 may be marked during pre-op (using the patient and possibly a stencil) with a marker, plastic piece, shrink band, tape, sleeve, or some other method, allowing the depth to be personalized for the patient. The stylet 2800 can be used as a stencil to mark the insertion point for patients during pre-op. The stylet 2800 may include an adjustable sleeve that indicates the total depth the stylet has traveled. The stylet 2800 may include a metal rod coated in plastic for easier marking addition. The stylet 2800 may include plastic bands overmolded into indents on the stylet as a marking method. The stylet 2800 may include different markings for when to start slowing down the stylet insertion, when a user should stop the stylet insertion, and when a user must stop the stylet insertion.

The stylet 2800 may include a removable handle that is then clamped over the stylet and catheter at the point where insertion should stop for that patient, preventing over-insertion. The stylet 2800 may include sensors or transmitters to help locate the stylet and catheter (e.g., inductive, capacitive, RF, ultrasound, laser, mechanical proximity sensors, liquid sensors, magnetic etc.). The stylet 2800 may emit a signal (e.g., vibrations, noise, clicks, light, etc.) that the inserter can sense on the external surface of the body to confirm correct placement. The signal could be generated by the stylet itself (e.g., a small motor in the handle that generates a vibration, or a speaker that makes a noise in the tip). The stylet 2800 may transmit a signal (e.g., vibrations, noise, clicks, etc.,) that the inserter can sense on the external surface of the body to confirm correct placement. The signal may be generated external to the stylet and then transmitted through the stylet (e.g., the user may tap the handle of the stylet with a hard object while feeling through the skin for the vibration at the internal tip). The stylet 2800 may emit a signal (e.g., vibrations, noise, clicks, etc.) that the inserter can sense through the stylet to confirm correct placement (e.g., a gently vibrating stylet—when the tip reaches the bladder the vibrations are dampened at the handle, or when the tip reaches the pubic symphysis bone the vibrations are magnified at the handle). The stylet 2800 may transmit a signal (e.g., vibrations, noise, clicks, etc.) that the inserter can sense through the stylet to confirm correct placement. The signal may be generated external to the stylet and then transmitted through the stylet (e.g., the user may tap the external end of the stylet with a hard object while feeling through the handle whether the tap is transmitted or dampened, showing whether the stylet is touching a soft area [e.g. bladder] or a hard area [e.g. bone]).

The stylet 2800 may include a magnetic tip or magnetic portion. Together with an external magnet, the stylet can be moved or placed in the correct position. The stylet 2800 may include a magnetic tip or magnetic portion. Together with an external magnet or sensor, the user can sense whether the stylet is in the correct position

### CONCLUSION

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A safety needle apparatus for insertion of a peritoneal dialysis catheter, the apparatus comprising:
a shaft including a first end and a second end, the first end including a sharp bevel;
a handle connected to the second end of the shaft, the handle including an engagement lever moveable between a lock position and an unlock position;
a guidewire lumen including a first end and a second end, the first end being aligned with the first end of the shaft, the guidewire lumen configured to receive a guidewire for placement of the peritoneal dialysis catheter; and
a blunt tip located within the shaft at the first end, the blunt tip connected to the engagement lever of the handle by a retraction mechanism,
wherein the engagement lever of the handle is configured to prevent retraction of the blunt tip into the shaft when the engagement lever is moved to the lock position.

2. The apparatus of claim 1, wherein the retraction mechanism includes a spring that causes the blunt tip to extend past the sharp bevel when the engagement lever is moved from a sharp bevel exposure position to a blunt tip exposure position.

3. The apparatus of claim 2, wherein moving the engagement lever from the blunt tip exposure position to the sharp bevel exposure position enables the spring to be pushed into a compressed state from resistance of the blunt tip contacting tissue.

4. The apparatus of claim 1, wherein the shaft has a diameter that is equal to or within 3 millimeters of a diameter of the peritoneal dialysis catheter.

5. The apparatus of claim 1, wherein the first end of the shaft includes an echogenic texture for ultrasound visualization.

6. The apparatus of claim 1, wherein the handle includes a sharp bevel indicator that is configured to provide a visual indication when the engagement lever is in a sharp bevel exposure position.

7. The apparatus of claim 1, wherein the guidewire lumen is connected to an exterior of the shaft and an exterior of the handle.

8. The apparatus of claim 1, wherein the guidewire lumen is positioned inside the shaft and the handle.

9. The apparatus of claim 1, wherein at least a portion of the blunt tip includes a transparent material, and
wherein the blunt tip includes an endoscope camera.

10. The apparatus of claim 9, wherein the endoscope camera is communicatively coupled to at least one of a tablet computer, a smartphone, or workstation, or a display monitor.

11. The apparatus of claim 1, wherein the blunt tip includes a blunt obturator or an inner shaft and the sharp bevel includes a sharp cannula or an outer shaft.

12. A catheter insertion method comprising:
causing an access hole to be created through skin, fat tissue, and muscle to a peritoneal cavity of a patient using a puncture device having a first diameter, the puncture device including a retractable blunt tip that exposes a sharp tip when retracted;
causing a guidewire to be routed into the peritoneal cavity through the puncture device after the blunt tip is extended past the sharp tip;
causing the puncture device to be removed after the guidewire is routed into the peritoneal cavity;
causing a first end of a catheter to be inserted through the access hole into the peritoneal cavity along the guidewire, the catheter having a second diameter;
causing the guidewire to be removed after insertion of the first end of the catheter;
causing a surgical sealant to create a seal between a portion of the catheter and adjacent muscle at the access hole;
causing a tunneling device to create a pathway through the fat tissue under the skin of the patient, the tunneling device including a first end with a retractable sharp tip or a blunt tip and a second end connected to a removable handle, the second end connectable to a second end of the catheter to pull the catheter through the pathway when the handle is removed;
causing a piercing tool having a needle in a middle of an alignment cup to align with the first end of the tunneling device to sandwich a portion of the skin therebetween;
causing the needle of the piercing tool to pierce the portion of the skin externally to create a catheter exit hole;
causing the tunneling device and the second end of the catheter to exit the patient at the catheter exit hole; and
causing the second end of the catheter to be connected to peritoneal dialysis tubing for a peritoneal dialysis treatment.

13. The method of claim 12, wherein the puncture device includes at least one of a mosquito hemostat, a single safety needle, a dual lumen safety needle, single lumen trocar, or a duel lumen trocar.

14. The method of claim 12, wherein the blunt tip of the puncture device is configured to cover the sharp tip after the puncture device creates the access hole to the peritoneal cavity.

15. The method of claim 12, wherein the first diameter of the puncture device and the second diameter of the catheter are between 3 millimeters (“mm”) and 15 mm.

16. The method of claim 15, wherein the first diameter of the puncture device is equal to the second diameter of the catheter.

17. The method of claim 15, wherein the first diameter of the puncture device is between 0.5 mm and 3 mm shorter than the second diameter of the catheter.

18. The method of claim 12, wherein the surgical sealant includes at least one of fibrin, albumin, a synthetic sealant, or combinations thereof.

19. The method of claim 12, wherein the puncture device includes a first lumen to enable the guidewire the pass through and a second lumen to enable an endoscope to pass through to the peritoneal cavity to provide visualization for placement of the guidewire.

20. The method of claim 12, further comprising causing a sheath or a dilator to be inserted into the access hole after the puncture device is removed for catheter insertion
